(19) United States
(12) Patent Application Publication
(10) Pub. No.: US 2026/0125428 A1
(43) Pub. Date: May 7, 2026

(54) SYNTHETIC TRIPLEX PEPTIDE NUCLEIC ACID-BASED INHIBITORS FOR CANCER THERAPY

(71) Applicant: University of Connecticut, Farmington, CT (US)

(72) Inventors: Raman Bahal, Glastonbury, CT (US); Shipra Malik, Storrs, CT (US); Vikas Kumar, Wilimantic, CT (US)

(21) Appl. No.: 18/858,884

(22) PCT Filed: Apr. 20, 2023
(86) PCT No.: PCT/US2023/066007

§ 371 Date: Oct. 22, 2024

Related U.S. Application Data
(60) Provisional application No. 63/334,839, filed on Apr. 26, 2022.

(51) Int. Cl. C07K 14/00; A61K 38/00; A61K 45/06; A61P 35/00; C12N 15/113

(52) U.S. Cl. CPC C07K 14/003; A61K 45/06; A61P 35/00; C12N 15/1135; A61K 38/00; C07K 2319/09; C12N 2310/3181

(57) ABSTRACT

A novel peptide nucleic acid (PNA) oligomer capable of forming a PNA/DNA/PNA triplex when binding to its target genomic DNA is described. An PNA oligomer directed to C-Myc oncogene was capable of binding the target DNA and effectively inhibit the transcription of the gene both in vitro as well as in vivo without causing any toxicity. Methods of making and using the novel PNA oligomer for targeting other genomic DNA are described.

### CROSS-REFERENCE TO RELATED APPLICATIONS

This application is a National Stage Application of International Patent Application No. PCT/US2023/066007, filed 20 Apr. 2023, which claims priority to, and the benefit of, U.S. Provisional Application 63/334,839, filed on Apr. 26, 2022, each of which is incorporated by reference herein in its entirety.

### SEQUENCE LISTING

The Instant Application contains a Sequence Listing which has been submitted electronically in XML format and is hereby incorporated by reference in its entirety. Said XML copy, created on 12 Jun. 2025, is named “UCT0293US2_Sequence_Listing.xml” and is 31,952 bytes in size.

### BACKGROUND

Among several oncogenes dysregulated in cancer, transcription factors are the most challenging to inhibit due to their localization in the nucleus. Current therapeutics for effective inhibition of undruggable proteins focuses upon targeting the mRNA present in the cytoplasm to inhibit the translation of proteins. The field of RNA medicine has gained momentum with the approval of three RNA interference (RNAi) based drugs; Onpattro® (patisiran), Givlaari® (givosiran), and Oxlumo™ (lumasiran) along with the success of antisense oligonucleotides; Tegsedi® (inotersen), Vyondys 53 (golodirsen) and Milasen. Even though mRNA can be targeted to prevent protein synthesis, there are often multiple mRNA molecules present in the cytoplasm and more mRNA is continuously being transcribed from the DNA. Further, tissue specific delivery of antisense molecules to sites other than the liver is challenging. Moreover, no antisense based drug has been approved for the treatment of cancer. Although small molecules have been used to target the genomic DNA for cancer therapy, they bind non-specifically and are associated with severe toxicological issues. Hence, there is a need to develop clinically translatable platforms for sequence specific targeting of genomic DNA.

### BRIEF SUMMARY OF THE INVENTION

The present disclosure provides a platform for targeting genomic DNA at the transcription activation site of oncogenes known to cause differentiation and proliferation of a wide range of tumors like breast cancer, hematological malignancies, prostate cancer, gastric cancer, lung, liver, pancreatic cancer and glioblastomas. Such a platform can be used as an adjunct therapy with chemotherapeutic drugs for treatment of resistant as well as relapsed tumors. In addition, a genomic DNA targeting strategy can also be utilized for inhibiting the transcription of genes involved in the pathophysiology of other non-malignant disorders. Moreover, the scope of this technology can be further expanded by targeting the genomic DNA to induce the activation of transcription leading to the proteins essential for normal physiological functions.

In an aspect, a peptide nucleic acid (PNA) oligomer that forms a PNA/DNA/PNA triplex invasion complex with a homopurine region of a target DNA has the formula:

5′-first PNA segment-flexible linker 1-second PNA segment-3′

wherein the first PNA segment is complementary to a homopurine stretch in the DNA, the second PNA segment is complementary to a region of the DNA including the homopurine stretch, wherein a nuclear localization signal (NLS) peptide is conjugated to the oligomer at the C-terminus, N-terminus, or both C- and N-termini, wherein the first PNA segment and the second PNA segment form the PNA/DNA/PNA triplex structure with the DNA, and wherein the DNA is genomic DNA. In another aspect, the PNA is a gamma-modified PNA with one or more gamma-modified monomer units.

In another aspect, a method for inhibiting transcription of a target gene involved in health disorders in a subject, comprising providing to a cell of the subject in vivo or ex vivo the above-described PNA oligomer, wherein the binding of the PNA oligomer to the targeted DNA region reduces expression of the targeted gene. In yet another aspect, a factor that opens chromatin DNA structure making the target DNA more accessible for PNA invasion is also administered.

### BRIEF DESCRIPTION OF THE DRAWINGS

FIGS. 1A-C show the location and sequence of the target site and the sequences of the indicated PNAs.

(1A) Organization of the C-MYC gene and position of the target sites. The gene map of C-MYC (GeneBank: AH002904.2; SEQ ID NO: 1) oncogene in humans.

(1B) The sequence of the target site 1 (SEQ ID NO: 2) and target site 2 (SEQ ID NO: 3) upstream of the promoter 1 and promoter 2 respectively.

(1C) Sequence of JPNAs designed to target the selected sites.

X₁CCTTCCCCACCCTCCCCACCCTCX₂, wherein X₁ is JJJJTTJJ-linker-, wherein J is pseudoisocytosine and the linker is 11-Amino-3,6,9-Trioxaundecanoic Acid, DCHA) represented as -OOO-, wherein X₂ is K-linker-SEQ ID NO: 5 (SEQ ID NO: 4)

X₃TCCCTCCCTCCGTTCTTTTTCCCX₂, wherein X₃ is JJJTJJJT-linker, wherein J is pseudoisocytosine and the linker is 11-Amino-3,6,9-Trioxaundecanoic Acid, DCHA) represented as -OOO-, wherein X₂ is K linker-SEQ ID NO: 5, wherein the linker is 11-Amino-3,6,9-Trioxaundecanoic Acid, DCHA) represented as -OOO- (SEQ ID NO: 6)

X₃TCCCTCCCTCCGTTCTTTTTCCCX₄, wherein X₃ is JJJTJJJT-linker, wherein J is pseudoisocytosine and the linker is 11-Amino-3,6,9-Trioxaundecanoic Acid, DCHA) represented as -OOO-, wherein X₄ is K (SEQ ID NO: 7)

X₅CCTCCCTTCTTCCTTCTCCCTTGX₂, wherein X₅ is TJJJJJJTJ-linker, wherein J is pseudoisocytosine and the linker is 11-Amino-3,6,9-Trioxaundecanoic Acid, DCHA) represented as -OOO-, wherein X₂ is K linker-SEQ ID NO: 5, wherein the linker is 11-Amino-3,6,9-Trioxaundecanoic Acid, DCHA) represented as -OOO- (SEQ ID NO: 8)

X₃TCCCTCCCCTCCCTTCTTTTTCCX₂, wherein X₃ is JJJTJJJT-linker, wherein J is pseudoisocytosine and the linker is 11-Amino-3,6,9-Trioxaundecanoic Acid, DCHA) represented as -OOO-, wherein X₂ is K linker-SEQ ID NO: 5, wherein the linker is 11-Amino-3,6,9-Trioxaundecanoic Acid, DCHA) represented as -OOO- (SEQ ID NO: 9)

X₃TCCCTCCCTCCGTTCTTTTTTCCCX₆, wherein X₃ is JJJTJJJT-linker, wherein J is pseudoisocytosine and the linker is 11-Amino-3,6,9-Trioxaundecanoic Acid, DCHA) represented as -OOO-, wherein X₆ is K-linker-Label, wherein the linker is 11-Amino-3,6,9-Trioxaundecanoic Acid, DCHA) represented as -OOO-, and the label is TAMARA (SEQ ID NO: 10)

X₃TCCCTCCCTCCGTTCTTTTTTCCCX₇, X₃ is JJJTJJJT-linker, wherein J is pseudoisocytosine and the linker is 11-Amino-3,6,9-Trioxaundecanoic Acid, DCHA) represented as -OOO-, X₇ is K linker-SEQ ID NO: 5-linker-label, wherein the linker is 11-Amino-3,6,9-Trioxaundecanoic Acid, DCHA) represented as -OOO-, and the label is TAMARA (SEQ ID NO: 11)

Nuclear localization signal peptide sequence is VKRKKKP (SEQ ID NO: 5). \γPNA-5-NLS is complementary to the mouse C-Myc target site upstream of promoter 2. OOO indicates polyethylene glycol linker. γPNA6 and γPNA7-NLS are conjugated with 3′ carboxy-tetramethylrhodamine dye (TAMRA).

FIG. 2 shows an in vitro binding study of γPNA1-NLS, γPNA2-NLS, ScrγPNA4-NLS with double stranded DNA (dsDNA1 and dsDNA2 containing target site for γPNA1-NLS and γPNA2-NLS respectively). γPNA-NLS were incubated with the target dsDNA at different concentrations in 10 mM sodium phosphate buffer at 37° C. for 17 hours. The dsDNA fragments were separated on a PAGE gel followed by visualization using SYBR™ gold.

FIGS. 3A-C show cellular uptake of indicated PNAs. (3A) A confocal image of HeLa cells showing cellular and nuclear uptake of γPNA7-NLS containing TAMRA at 2 μM dose and after 24 h of treatment. (3B) A confocal image of U2932 cells showing cellular and nuclear uptake of γPNA7-NLS containing tetramethylrhodamine (TAMRA) at 2 μM dose and after 24 h of treatment. (3C) A histogram representing flow cytometry results for cellular uptake of γPNA7-NLS in U2932 cells after incubation at different doses for 24 h. The scale bar represents 10 μm.

FIGS. 4A-F show effect of cellular uptake of indicated PNAs. (4A) Expression levels of C-Myc oncogene in U2932 (DLBCL), Raji and Daudi cells (Burkitt's lymphoma) in comparison to the peripheral blood mononuclear cells (PBMC). (4B) Cell viability of U2932 cells after 24 h treatment with indicated PNAs at different doses. (4C) Cell viability of Raji cells after 24 h treatment with indicated PNAs at different doses. (4D) Gene expression levels of C-Myc oncogene and its downstream targets including MCL1 and EZH2 in U2932 cells after 24 h of treatment with γPNA1-NLS, γPNA2-NLS, and ScrγPNA4-NLS at 8 μM. (4E) Gene expression levels of C-Myc oncogene and its downstream targets including BCL2 and EZH2 in Raji cells after 24 h of treatment with γPNA1-NLS, γPNA2-NLS, and ScrγPNA4-NLS at 8 μM. (4F) Gene expression levels of C-Myc in U2932 cells after 24 h treatment with γPNA2-NLS and ScrγPNA4-NLS at indicated doses. The results in all graphs are represented as mean±SEM (n>3). *p<0.05, **p<0.01, ***p<0.001, ****p<0.0001.

FIGS. 5A-D show results of in vivo administration of indicated PNAs. (5A) IVIS® images of xenograft mice bearing tumors at indicated time points after systemic administration of PNA-TAMRA and γPNA7-NLS at 5 mg/kg. (5B) IVIS® imaging of organs harvested from xenograft mice treated with PNA-TAMRA and γPNA7-NLS after 6 h of systemic administration. (5C) Quantification of fluorescence from the organs harvested from PNA-TAMRA and γPNA7-NLS treated xenograft mice. (5D) Confocal microscopy images of tumor and kidney tissue cryo-sections from PNA-TAMRA and γPNA7-NLS treated U2932 xenograft mice. Scale bar, 30 μm.

FIGS. 6A-C show effect of systemic administration of indicated PNAs. (6A) IVIS® images of xenograft mice bearing tumors at indicated time points after systemic administration of γPNA7-NLS and γPNA6 at 5 mg/kg. (6B) IVIS® imaging of organs harvested from control, γPNA7-NLS, and γPNA6 treated mice after 24 hours of systemic administration. (6C) Confocal imaging of tumor, liver, and kidney sections obtained from xenograft mice after 24 h of treatment with γPNA7-NLS and γPNA6. Scale bar is 30 μm.

FIGS. 7A-C show survival data of mice after systemic administration of indicated PNAs. (7A) Graph representing the tumor volume of U2932 xenografts after systemic treatment with indicated PNAs (n≥4 mice per group). PNAs were administered systemically on day 1, 4, 7, and 10 (n≥4 mice per group). (7B) The survival of U2932 derived xenograft mice after treatment with γPNA2-NLS, γPNA3, and ScrγPNA4-NLS at 5 mg/kg dose in comparison to the control (saline) mice. (7C) Complete blood count analysis of mice from different groups at the end of the study. RBC: red blood cells; WBC: white blood cells; HGB: hemoglobin; PLT: platelets; HCT: hematocrit; MCH: mean corpuscular volume. (D) Blood chemistry analysis of mice from different groups at the end of the study. ALT: alanine transaminase; AST: aspartate transaminase; BUN: blood urea nitrogen; LDH: Lactate dehydrogenase. Results are presented as mean±SEM (n≥4).

FIGS. 8A-D show organ distribution after intravenous and subcutaneous administration of indicated PNAs. (8A) The organ distribution of γPNA-NLS TAMRA after intravenous (IV) and subcutaneous (SC) administration in transgenic mice model of B-cell lymphoma (Ep-myc). γPNA-NLS TAMRA was administered at 5 mg/kg via IV and SC route and organ distribution was determined via IVIS imaging after 24 h. (8B) Confocal microscopy images of different lymph nodes (LNs) after IV and SC injection of γPNA-NLS TAMRA. Blue indicates DAPI and red indicated TAMRA. Scale bar, 30 μm. (8C) C-myc protein levels in brachial lymph nodes of γPNA5-NLS treated mice in comparison to the control (untreated mice). N=4 mice per group. (8D) C-myc protein levels in axillary lymph nodes of γPNA5-NLS treated mice in comparison to the control (untreated mice). N=4 mice per group. Transgenic or Ep-myc mice were treated with γPNA5-NLS at 60 mg/kg dose over 2 days and mice were euthanized on day 3 for analysis.

FIGS. 9A-D show cell viability after treatment with indicated PNAs. (9A) The viability of U2932 cells after treatment with γPNA1-NLS (8 μM), romidepsin (10 nM) and combination of γPNA1-NLS with romidepsin over 96 hours. (9B) The viability of U2932 cells after treatment with γPNA2-NLS (8 μM), romidepsin (10 nM) and combination of γPNA2-NLS with romidepsin over 96 hours. (9C) Fold change in C-Myc levels after combination of γPNA1-NLS and γPNA2-NLS with romidepsin in comparison to only romidepsin treatment. (9D) Fold change in BCL2 levels after combination of γPNA1-NLS and γPNA2-NLS with romidepsin in comparison to only romidepsin treatment. Results are presented as mean±SEM (n=4). ***p<0.001, ****p<0.0001.

FIGS. 10A-F show the viability of U2932 cells after indicated treatment. (10A) Viability of U2932 cells after treatment with γPNA2-NLS (μM), vorinostat (2.5 μM) and combination of γPNA2-NLS with vorinostat over 96 hours. (10B) The viability of U2932 cells after treatment with γPNA2-NLS (8 μM), valproic acid (2 mM) and combination of γPNA2-NLS with valproic acid over 96 hours. (10C) Fold change in C-Myc mRNA levels after combination of γPNA1-NLS, γPNA2-NLS and Scr-γPNA4-NLS (8 μM) with vorinostat (2.5 uM) in comparison to only vorinostat treatment in U2932 cells after 96 h. (10D) Fold change in C-Myc mRNA levels after combination of γPNA1-NLS, γPNA2-NLS and Scr-γPNA4-NLS with valproic acid (2 mM) in comparison to only valproic acid treated U2932 cells after 96 h. (10E) The viability of U2932 cells after treatment with CHOP (cyclophosphamide, vincristine, doxorubicin, prednisone) and combination of γPNA2-NLS with CHOP at different doses. CHOP dose 1 indicates doxorubicin: 0.162 mM, cyclosphosphamide: 4.1 mM, vincristine sulfate: 15.5 M, prednisone: 0.002 g/ml. Viability was measured by trypan blue assay after 24 h and 48 h of treatment. Results are presented as mean±SEM (n=3). *p<0.05, ***p<0.001, ****p<0.0001.

### DETAILED DESCRIPTION

Described herein is a genomic DNA targeting strategy for inhibiting the transcription of a target gene, for example a gene involved in disease, such as for cancer therapy. The novel strategy involves the use of triplex forming peptide nucleic acids (PNAs) which include a tail clamp (tcPNA) and one or more nuclear localization signals (tcPNA-NLS) to assist delivery of the tcPNA to the nucleus. The tcPNA can have one or more gamma-modified residues found to increase binding affinity to target DNA. Gamma-modified tcPNAs have never been explored for targeting genomic DNA. When this targeting strategy is used for targeting sites in the promoter regions of C-Myc oncogene, the gamma-tcPNA-NLS show cellular and nuclear uptake in vitro in both Burkitt lymphoma (Raji and Daudi) cells and Diffuse Large B-Cell Lymphoma (DLBCL) (U2932) cells, and a significant decrease in viability of both U2923 and Raji cells. There was a 50% decrease in the levels of C-Myc, as well as its downstream targets including MCL1, BCL2 and EZH2 after 24 hour treatment. Systemically administered gamma-tcPNA-NLS in xenograft mouse models bearing tumors resulted in reduced tumor growth and increased survival of mice. Administration of the gamma-tcPNA-NLS in a transgenic model of Burkitt lymphoma resulted in a substantial decrease in c-myc protein levels in the treated mice. A combination treatment of gamma-tcPNA-NLS with a histone deacetylation inhibitor showed synergistic activity.

In an aspect, a peptide nucleic acid (PNA) oligomer that forms a PNA/DNA/PNA triplex invasion structure, where the DNA is a target genomic DNA, and where the PNA oligomer has the formula: 5′-first PNA segment-flexible linker-second PNA segment-3′ wherein the first PNA segment is complementary to a homopurine stretch in the target DNA, the second PNA segment is complementary to a region of the target DNA including the homopurine stretch, wherein the first PNA segment and the second PNA segment form the PNA/DNA/PNA triplex invasion structure with the DNA.

As used herein, PNA is a synthetic form of a nucleic acid which lacks a net electrical charge along its protein-like backbone. Specifically, PNAs are molecules in which the phosphodiester backbone of an oligonucleotide is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that are similar to oligonucleotides but are achiral and neutrally charged molecules. PNAs are comprised of peptide nucleic acid monomer units. The heterocyclic bases can be any of the standard bases (uracil, thymine, cytosine, adenine and guanine) or any of the modified heterocyclic bases described below. The neutral backbone of PNAs decreases electrostatic repulsion between the PNA and target DNA phosphates.

PNAs are typically single stranded and can recognize and bind to a target nucleic acid, double-stranded DNA (dsDNA), through the formation of a double-duplex invasion complex that does not require prior denaturation of dsDNA. The PNA/DNA hybrids are formed by Watson-Crick hydrogen bonds, but the binding affinities are significantly higher than those of a corresponding oligonucleotide composed of DNA or RNA. As used herein, the PNA binds DNA sufficiently to prevent expression of the bound DNA.

As used herein, the PNA oligomer comprises two PNA molecules linked together by a linker of sufficient flexibility to form a single PNA molecule which forms the PNA/DNA/PNA invasion triplex structure with the DNA. An exemplary linker is between 1 and 10 units of 8-amino-3,6-dioxaoctanoic acid (referred to as an O-linker), 6-aminohexanoic acid, 8-amino-2, 6, 10-trioxaoctanoic acid, or 11-amino-3, 6, 9-trioxaundecanoic acid. Poly(ethylene) glycol monomers can also be used as PNA linkers. A PNA linker can contain multiple linker monomers in any combination.

The first PNA segment in the PNA oligomer can be a pyrimidine stretch, e.g., 3-10 pyrimidines, that hybridizes to a homopurine stretch on the target DNA, also referred to as a “clamp” or tail clamp (tc) added to the end of the Watson-Crick binding portion. The clamp binds portions of the target nucleic acid or DNA by Hoogsteen base-pairing. The PNA oligomer with the tail clamp (tcPNA) mediates a mode of binding to DNA that encompasses both triplex and duplex formation with the clamp PNA forming a triplex portion, the PNA/DNA/PNA triplex, in addition to the second segment's PNA/DNA duplex portion.

Preferably, both the Watson-Crick and Hoogsteen binding portions of the triplex forming molecules are substantially complementary to the target sequence. In some aspects, the Hoogsteen binding segment of the PNA oligomer includes one or more, chemically modified cytosines such as pseudocytosine, pseudoisocytosine, and 5-methylcytosine.

In an aspect, the first PNA segment comprises one more pseudoisocytosine units. In another aspect, the first PNA segment comprises only pseudoisocytosine units and thymidine units.

The second segment of the PNA oligomer is complementary to a region of the DNA including the homopurine stretch. Specifically, it forms Watson-Crick bonding with the target DNA to selectively bind to or hybridize with a predetermined target sequence, target region, or target site within an DNA such that a triple-stranded structure is formed. The nucleotide sequence of the second PNA oligomer segment is selected based on the sequence of the target sequence, the physical constraints to achieve binding of the oligonucleotide within the major groove of the target region, and preferably to have a low dissociation constant (Kd) for the oligonucleotide/target sequence.

In some embodiments, the PNA oligomer including a first Hoogsteen binding peptide nucleic acid (PNA) segment and a second Watson-Crick binding PNA segment preferably collectively total no more than 50 nucleobases in length.

In an aspect, depending on the length of the target DNA region, the second PNA segment can be the full length or a partial length of the target DNA region. In one aspect, the second PNA segment is about 7-10 nucleotides in length, about 5-12 nucleotides in length, about 7-15 nucleotides in length, about 10-20 nucleotides in length, about 7-30 nucleotides in length, and up to the full length of the target DNA region.

In one aspect, the PNA oligomer forming a PNA/DNA/PNA triplex is a gamma PNA (γPNA) with a tail clamp, or a γtcPNA.

In another aspect, one or both, preferably both, PNA segments comprises one or more gamma-modified monomer units, wherein the PNA backbone contains one or more modification at the gamma-position of the N-(2-aminoethyl) glycine unit. Gamma-modified PNAs (gamma PNA or γPNA) are capable of adopting a right-handed helical conformation, can improve solubility, reduce self-aggregation, bind to the target with high affinity and sequence specificity and result in more stable PNA-DNA hybrids. Gamma PNA modifications include serine modified, lysine modified, glutamic acid modified, or alanine modifications. In an aspect, the first, second or both PNA segments are serine modified gamma PNAs. The synthesis of gamma PNAs is described in U.S. Pat. No. 10,221,216, incorporated herein by reference for the disclosure of gamma PNA and methods of synthesis of gamma PNA.

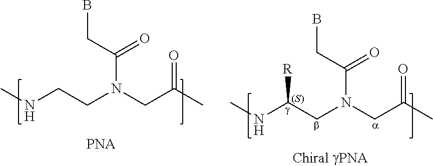

Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

Chemical modifications of the basic PNA structure are known and can be used. For example, fluorine-modified, cyclopentyl-modified, mini-peg-modified, guanidinium-modified, pyrrolidinyl-modified, and 2-aminopyridine-modified PNAs are known in the art and can be chosen for preparation of the PNA oligomer to improve cell permeability or increase DNA binding affinity.

Mini-Peg-containing γ-PNAs are described in U.S. Pat. No. 10,793,605, incorporated herein by reference for its disclosure of mini-PEG γ-PNAs and their methods of synthesis.

The PNA oligomers can also include other positively charged moieties to increase the solubility of the PNA, for increased cell permeability, and/or to increase the affinity of the PNA for the target DNA. Commonly used positively charged moieties include the amino acids lysine and arginine, although other positively charged moieties may also be useful. Lysine and arginine residues can be added to a tcPNA linker or can be added to the carboxy or the N-terminus of a PNA oligomer strand.

Exemplary modifications to PNA include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethyl glycine backbone skeleton with a negatively-charged scaffold; conjugation of high molecular weight polyethylene glycol (PEG) to one of the termini; fusion of PNA to RNA to generate a chimeric oligomer, redesign of the backbone architecture, conjugation of PNA to DNA or RNA. These modifications improve solubility but often result in reduced binding affinity and/or sequence specificity.

In an aspect, PNA is synthesized using monomer units by established solid-phase synthesis-based protocols known in the art.

In an aspect the first and second segments of the PNA oligomer bind to or hybridize to the target sequence under conditions of high stringency and specificity. Most preferably, the oligomer binds in a sequence-specific manner to the target DNA. Reaction conditions for in vitro triple helix formation of a PNA oligomer to a nucleic acid sequence vary from oligomer to oligomer, depending on factors such as oligomer length, the number of G:C and A:T base pairs, and the composition of the buffer utilized in the hybridization reaction. An oligomer substantially complementary to the target region of the nucleic acid molecule is preferred.

In another aspect, one or more nuclear localization signal (NLS) peptides is conjugated to the PNA oligomer. NLS peptide sequences mediate the transport of protein or DNA cargoes from the cytoplasm to the nucleus. NLS sequences are typically short peptide sequences composed of 4-8 basic amino acids, which generally contain 4 or more positively charged residues, e.g., lysine (K) and arginine (R). The classical and best understood NLS sequence is the SV40 NLS sequence derived from SV40 tumor antigen. The characteristic motif of NLS is defined as K(K/R)X(K/R), where X can by any residue. In the present aspect, the NLS is used to assist in transport of the PNA oligomer to the nucleus as well as improve cellular delivery. Without being held to theory, the limited cellular delivery of PNAs due to the hydrophobicity and neutral charge of the PNA can be improved by the presence of basic amino acids like lysine and arginine in the NLS peptide. Other NLS sequences are known in the art, for example, the NLS in vasopressin-activated calcium-mobilizing protein/cullin5, NLS from the chemokine receptor CXCR4, or from viral protein (VP1) of chicken anemia virus (CAV), NLS of C-terminus of nucleoplasmin, NLS of TP53-binding protein 1, among other NLS sequences capable of mediating transport of the PNA oligomer to the nucleus can be used. One or more NLS peptides can be conjugated to either the C or N terminus, or both. Methods of conjugating a peptide to a PNA are known in the art and described in the Examples below.

In one aspect, the target sequence is in a transcription control sequence of a gene, such as a promoter sequence, an enhancer sequence, a silencing sequence, a transcription-factor binding region, and the like. In an aspect, the target DNA sequence can induce or inhibit transcription of a gene. In another aspect, the gene is involved in a disease. In yet another aspect, the gene is an oncogene. In another aspect, the oncogene is a transcription factor or a tyrosine kinase. A nonlimiting list of oncogenes that can be targeted is provided in Table 1. As mentioned above, the novel platform is exemplified herein to target genomic DNA and inhibit the transcription of C-Myc oncogene. γtcPNAs have never been explored for targeting the genomic DNA.

TABLE 1
ONCOGENE TARGETS

| Oncogene class | Gene |
|---|---|
| Tyrosine Kinases | Epidermal growth factor receptor (EGFR) |
| | Platelet derived growth factor receptor |
| | Human epithelium growth factor receptor 2 |
| | (HER2) |
| | Vascular endothelial growth factor (VEGF) |
| | Cyclin dependent kinases (CDKN2 and CCND1) |
| Transcription Factors | Cellular myelocytomatosis (c-myc) |
| | Signal Transducer and Activator of Transcription |
| | (STATs) |
| | Nuclear Factor - κB |
| | Nuclear factor erythroid 2-related factor 2 (NRF2) |
| | Sry-related HMG box (SOX2) |
| | Heat Shock Factor (HSF1) |
| | Krüppel-Like factors (KLF 8) |
| | The CCCTC-binding factor (CTCF) |
| Others | B cell lymphoma -2 (BCL-2) |
| | mouse double minute 2 (MDM2) |
| | Programmed death -1 ligand (PD-L1) |
| | Cytotoxic T lymphocyte-associated antigen-4 |
| | (CTLA-4) |

The novel PNA oligomers described herein were tested by selecting the target sites close to the promoter regions of C-Myc oncogene and designing gamma modified tail clamp PNAs to inhibit transcription of C-Myc. Extensive bio-physical characterization of PNAs and their cellular uptake in multiple cell lines was performed. Delivery of the novel PNA oligomers in Burkitt lymphoma and diffuse large B cell lymphoma (DLBCL) cells as well as in a xenograft mice model of DLBCL established the efficacy of the novel PNA oligomers in inhibiting C-Myc transcription both in vitro and in vivo.

In an aspect, the PNA oligomer modifies the transcription or expression of a gene in a cell. Gene transcription is controlled by transcription factors and other proteins involved in producing mRNA from the gene. RNA polymerase II is an enzyme involved in initiating and mediating the transcription of a gene by binding to DNA at the gene promoter site(s) normally found upstream of the transcription start site of the mRNA. A promoter region is a sequence of DNA needed to turn a gene on or off. When RNA polymerase requires other proteins or factors to begin or continue transcription, reducing, inhibiting, or limiting the amount of mRNA that is produced from the gene can be achieved by hindering RNA polymerase, or assisting factors, from binding to DNA. This can be achieved by several methods, including introducing a PNA oligomer described herein, capable of binding to a target DNA sequence necessary for transcription, such as the transcription initiation at the promoter site. In some aspects, the promoter site is homopurine-homopyrimidine rich.

Gene maps describe the spatial arrangement of genes on a chromosome and shows regions important for transcription, such as promoter regions. For many organisms, such as a human, or mouse, the genome is well annotated. Genome browsers such as NCBI, Ensembl, and USCS are publicly available and can be used to retrieve desired target DNA sequences.

In some aspects, the PNA oligomer can have a sequence substantially complimentary to a target DNA, such that it allows formation of the triplex with the target DNA. In an aspect, depending on the length of the target DNA region, the second PNA segment can be the full length or partial length of the target DNA region. In one aspect, the PNA is about 7-10 nucleotides in length, about 5-12 nucleotides in length, about 7-15 nucleotides in length, about 10-20 nucleotides in length, about 7-30 nucleotides in length, and up to the full length of the target DNA region.

In an aspect the first and second segments of the PNA oligomer bind to or hybridize to the target sequence under conditions of high stringency and specificity. Most preferably, the oligomers bind in a sequence-specific manner to either strand of the double-stranded target DNA. Reaction conditions for in vitro triple helix formation of a PNA oligomer to a nucleic acid sequence vary from oligomer to oligomer, depending on factors such as oligomer length, the number of G:C and A:T base pairs, and the composition of the buffer utilized in the hybridization reaction. An oligomer substantially complementary to the target region of the nucleic acid molecule is preferred.

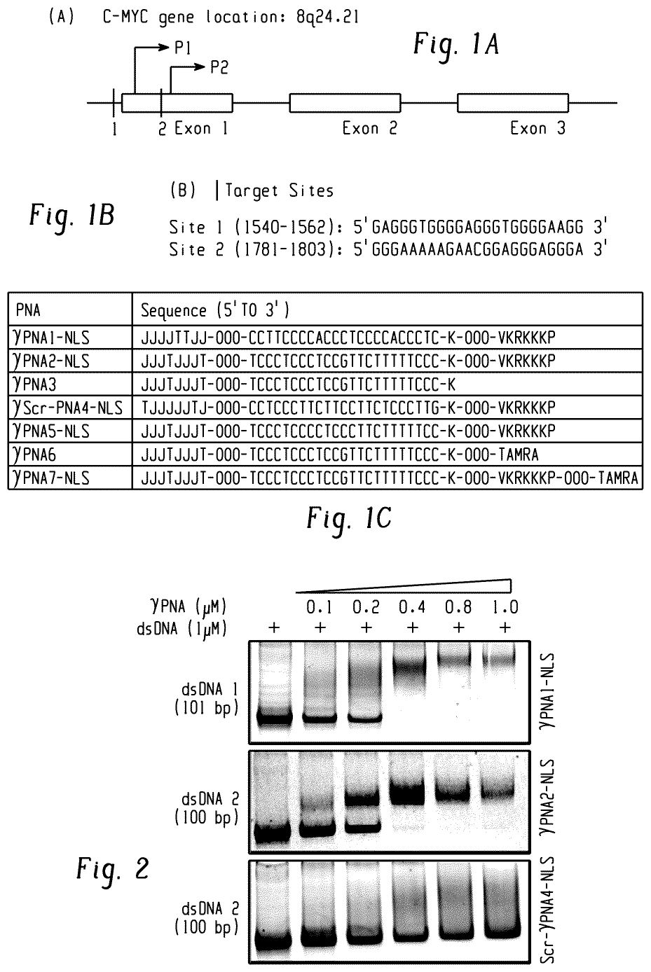

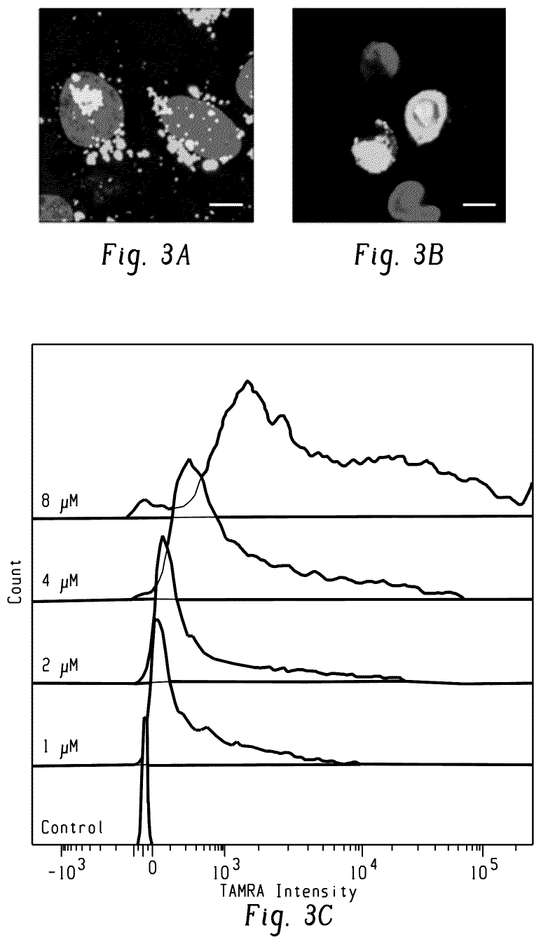

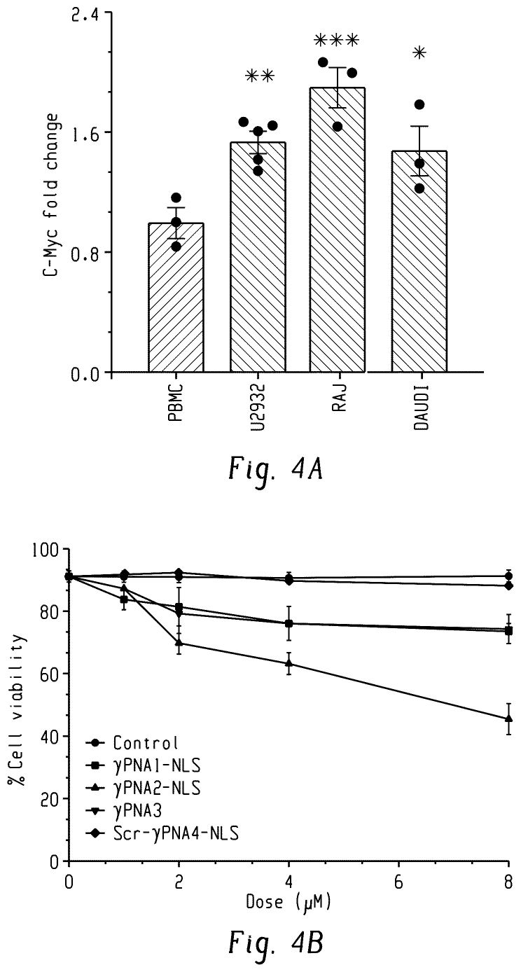

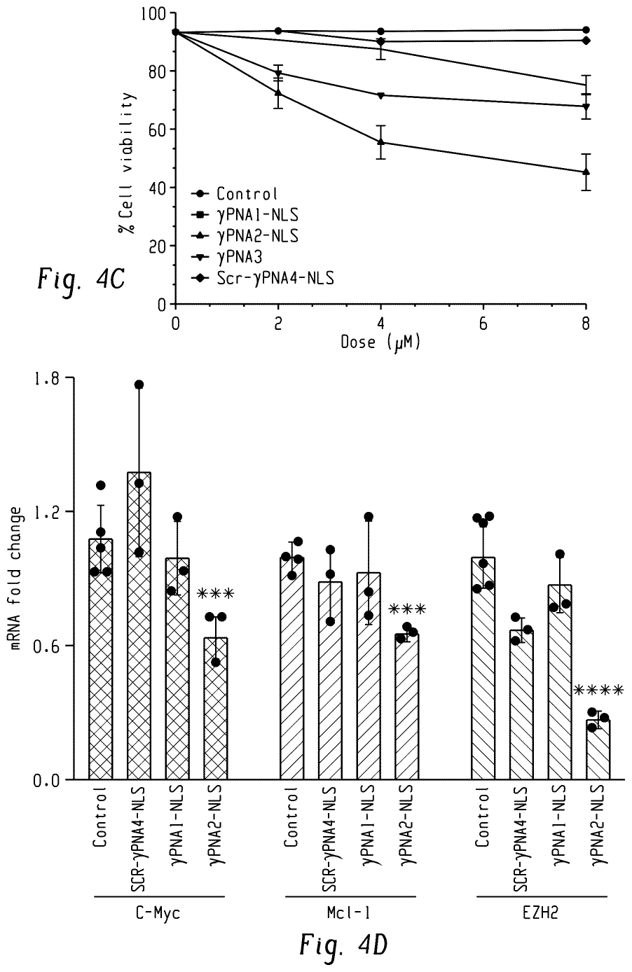

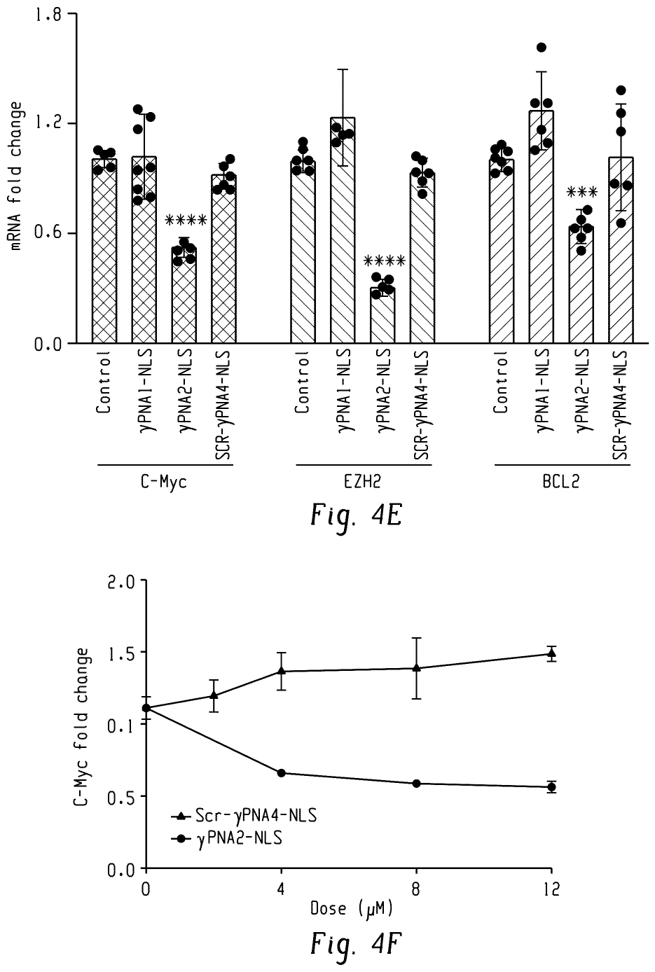

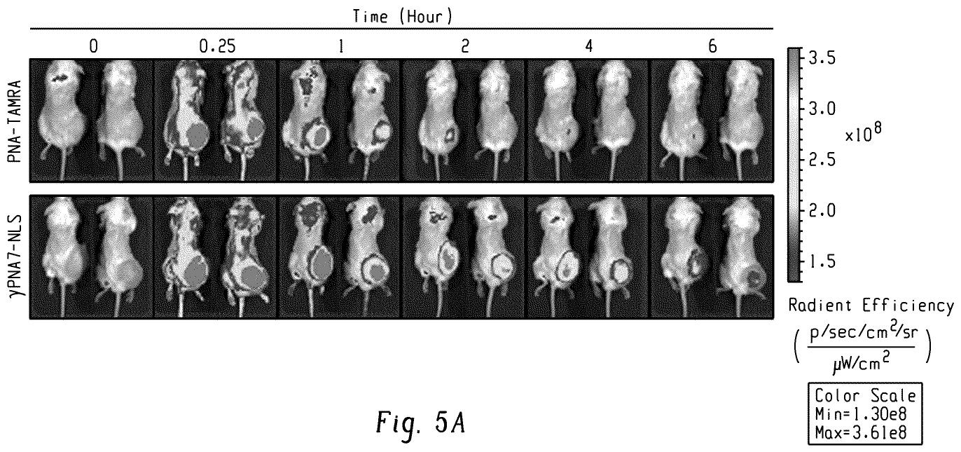

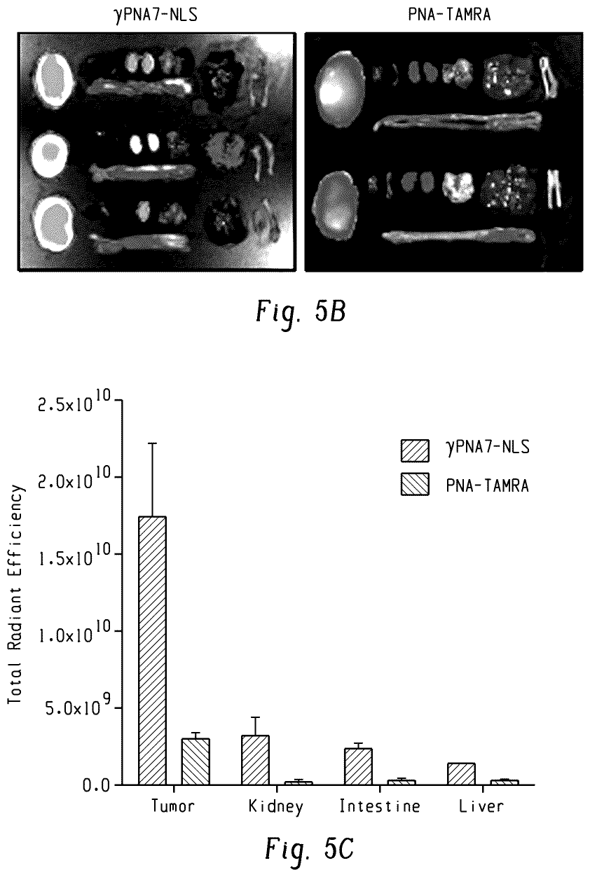

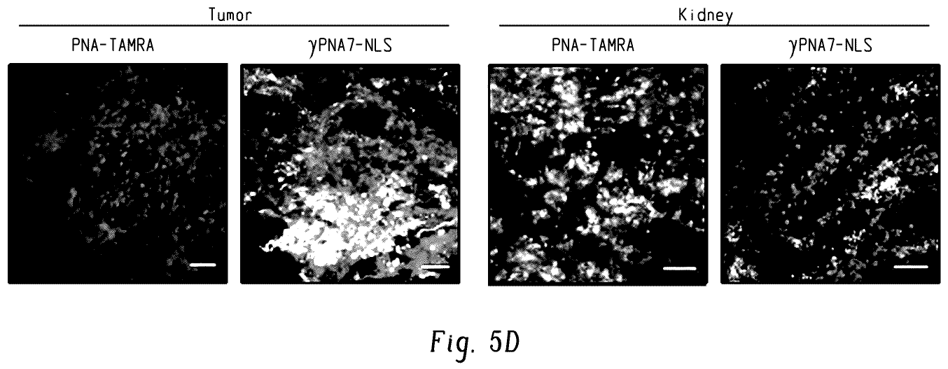

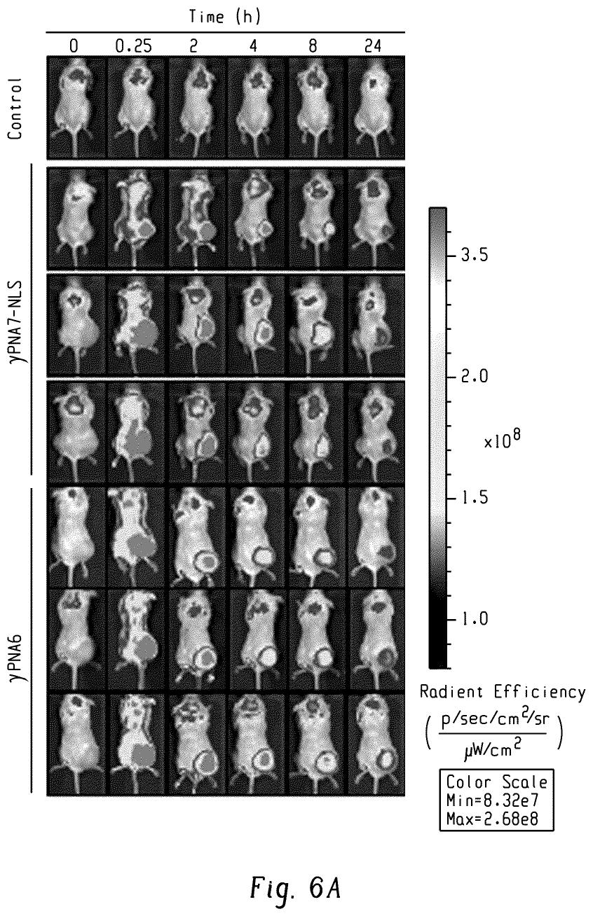

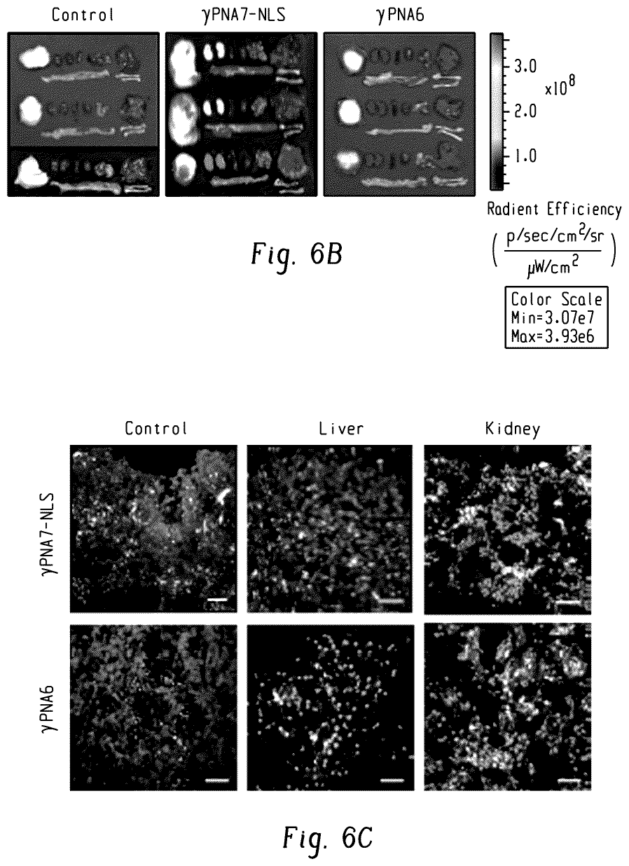

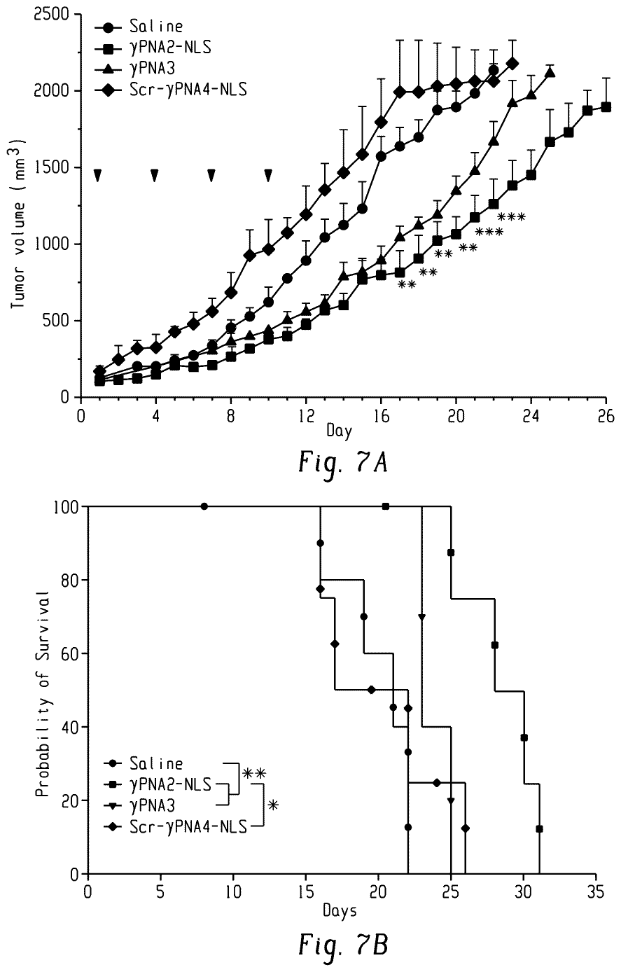

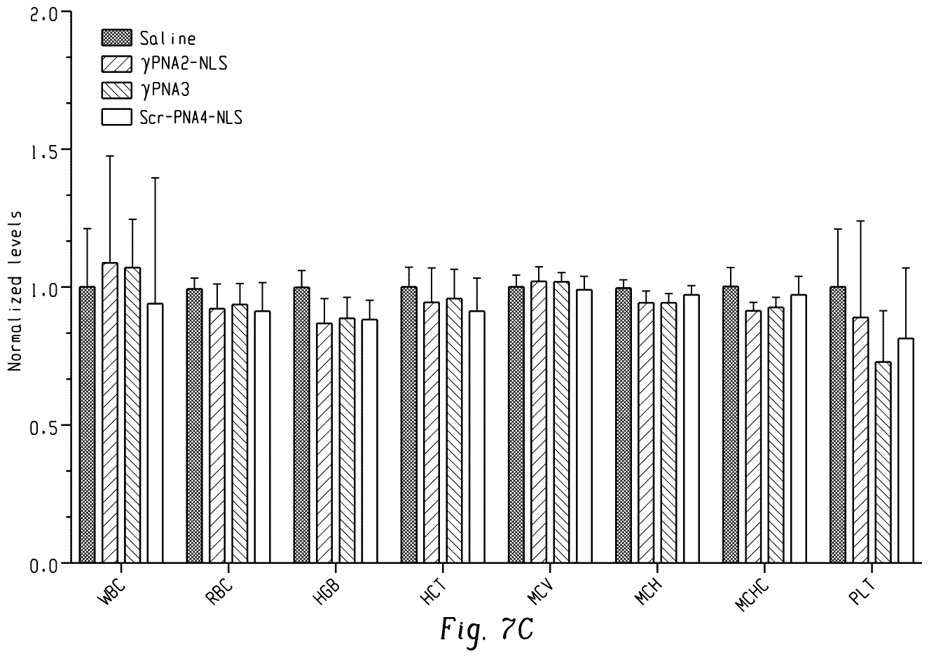

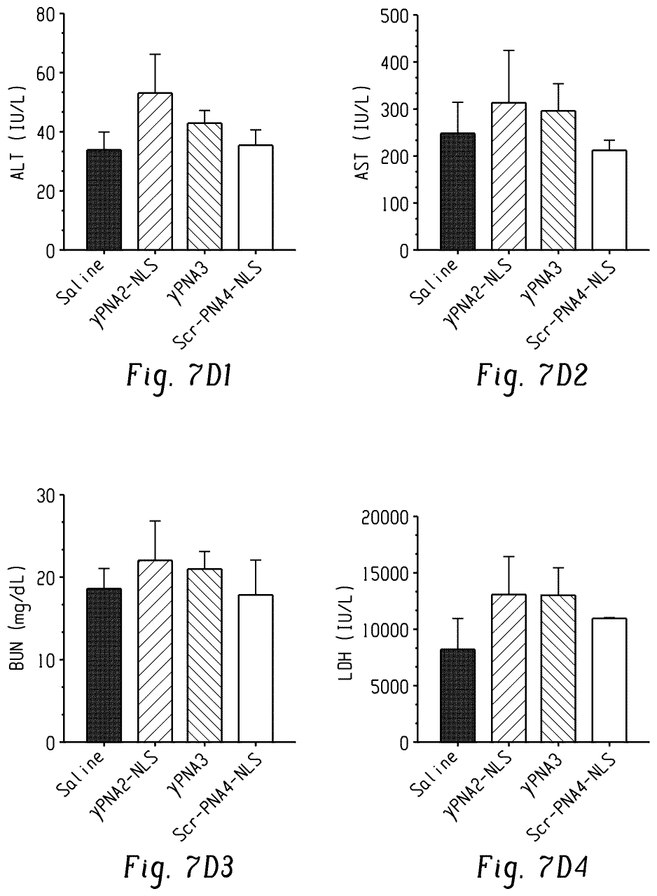

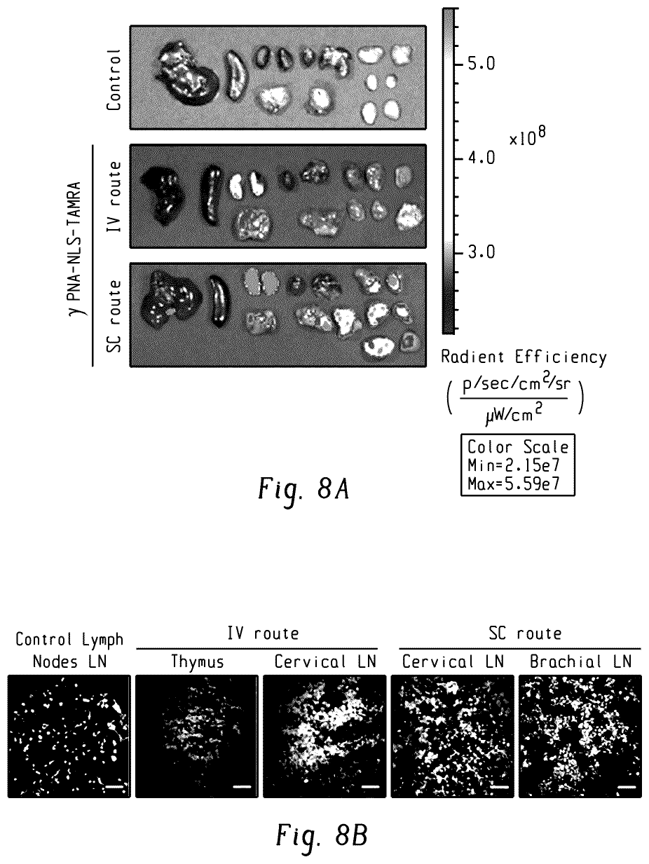

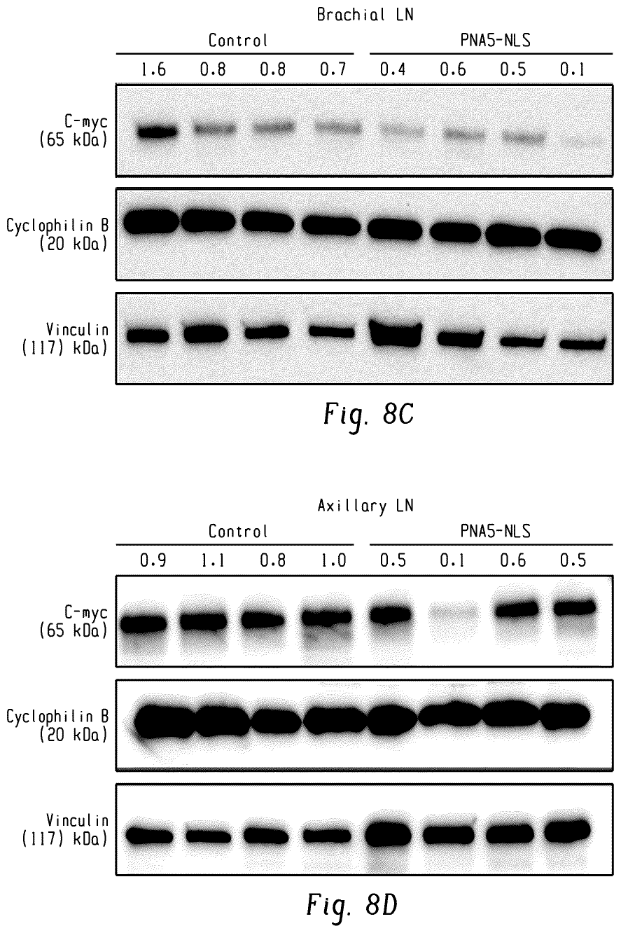

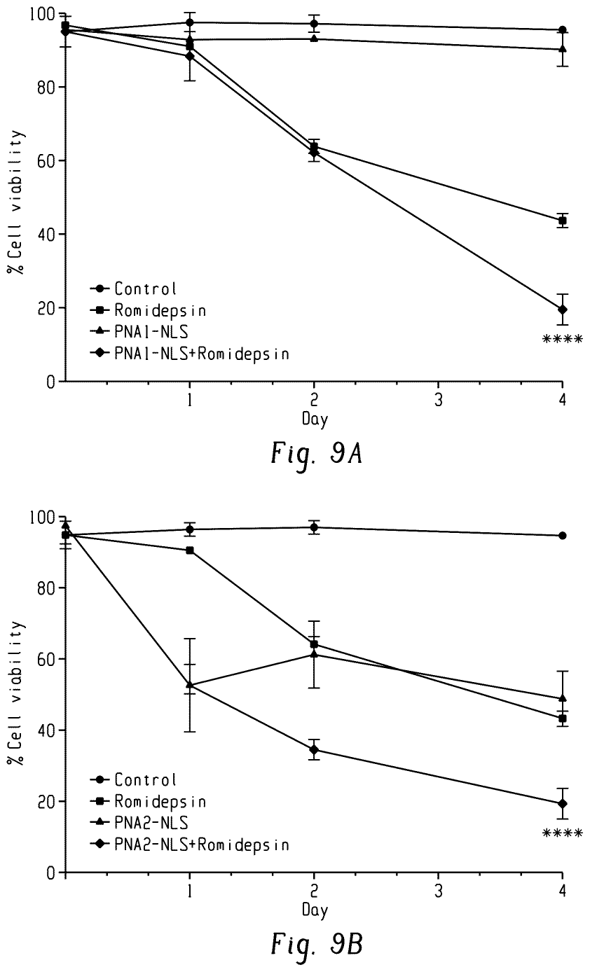

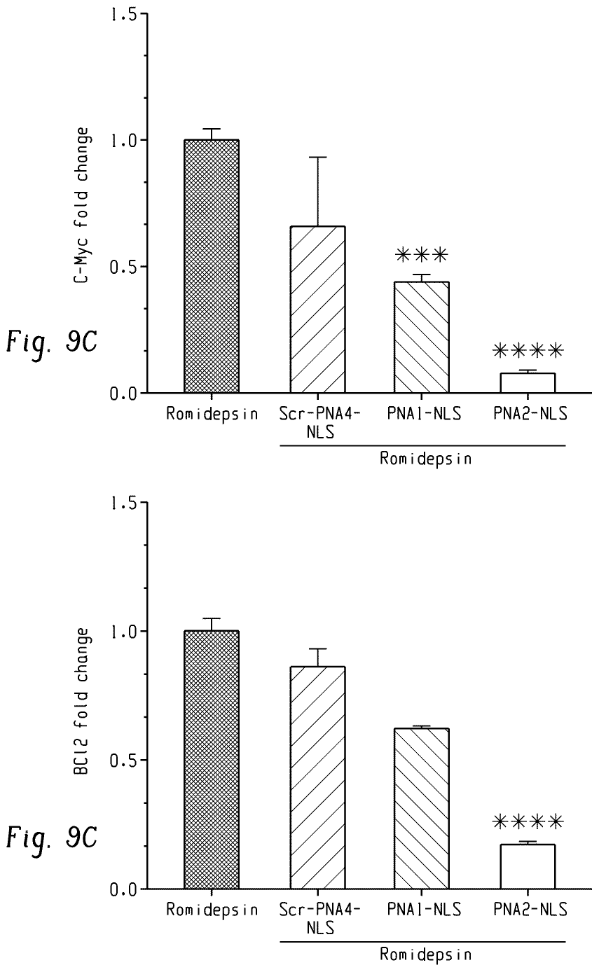

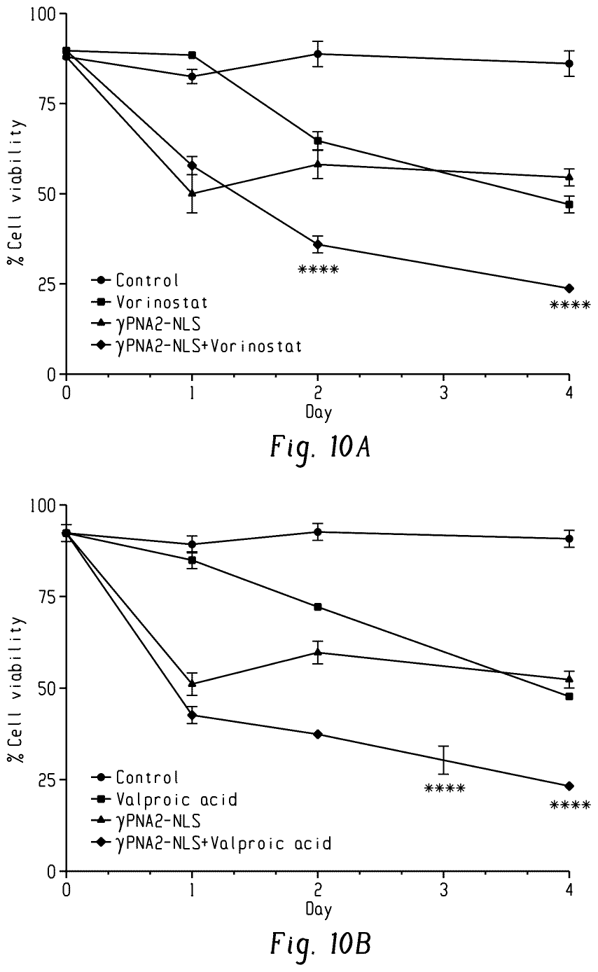

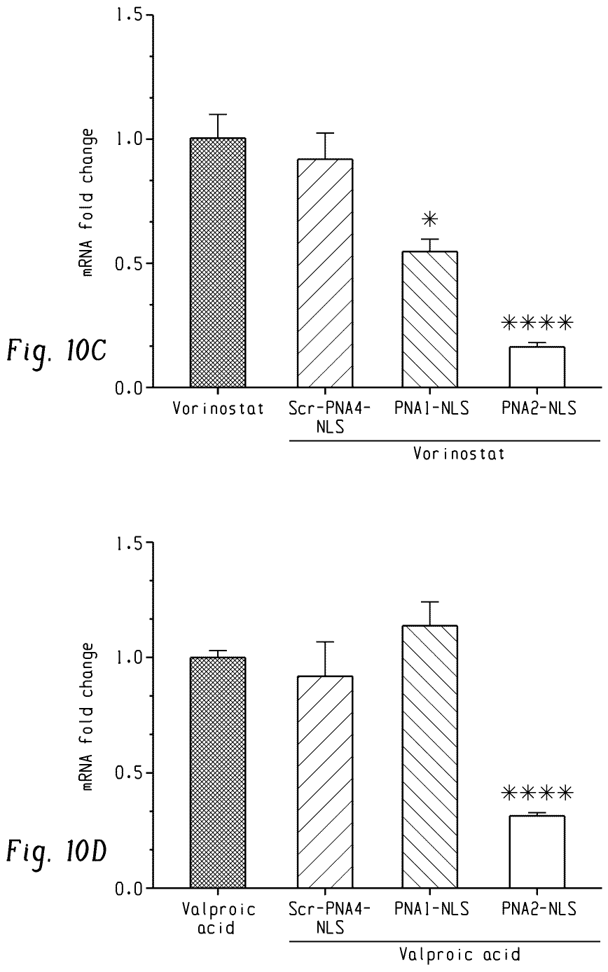

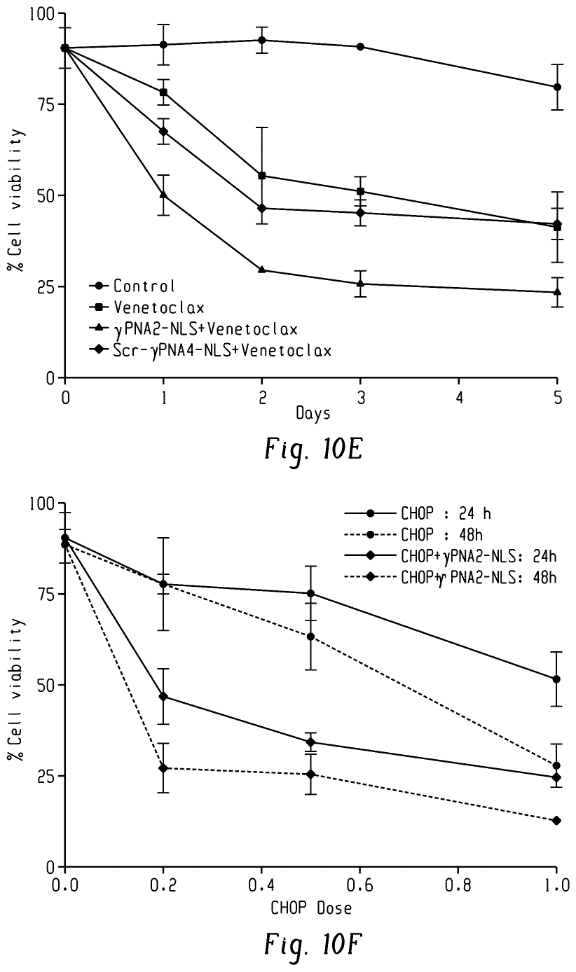

As exemplified herein below and shown in FIG. 1, C-Myc oncogene transcription was regulated by introducing a PNA oligomer described herein. The C-Myc oncogene, located on chromosome 8, consists of three exons. Exon 1 is non-coding while exon 2 and exon 3 encode for the c-myc protein. There are four transcriptional promoters reported for regulating the expression of c-myc. Exon 1 contains two promoter regions (P1 and P2) which regulate transcription initiation. Complementary PNA oligomers were designed to target sites containing homopurine region upstream of both P1 and P2 were selected.

Accordingly, in some embodiments, a C-Myc Exon 1 target DNA upstream of the promoter 1 can comprise the sequence of Site 1 (Site 1, nucleotide 1540-1562 of the C-Myc genomic sequence GeneBank:AH002904.2), 5′GAGGGTGGGGAGGGTGGGGAAGG 3′ (SEQ ID NO: 2) or a variant thereof that retains the ability of binding to Site 1 target DNA. Gamma-PNA-NLS oligomer with a tail clamp sequence 5′ JJJJTTJJ 3′ where ‘J’ is pseudoisocytosine, a polyethylene glycol linker 5′ OOO 3′, a NLS peptide 5′ VKRKKKP 3′ (SEQ ID NO: 5), capable of binding to Site 1 target DNA can comprise a sequence 5′ JJJJTTJJ-OOO-CCTTCCCCACCCTCCCCACCCTC-K-OOO-VKRKKKP 3′(SEQ ID NO:4), or a portion or variant thereof that retains the ability to bind to the target DNA.

In another embodiment, a C-Myc Exon 1 target DNA upstream of promoter 2 can comprise the sequence of Site 2 (Site 2, nucleotide 1781-1803 of the C-Myc genomic sequence GeneBank:AH002904.2), 5′ GGGAAAAAGAACGGAGGGAGGGA 3′ (SEQ ID NO:3) or a variant thereof capable of binding to Site 2 target DNA. Gamma-PNA-NLS oligomer with a tail clamp sequence 5′ JJJTJJJT 3′ where ‘J’ is pseudoisocytosine, a polyethylene glycol linker 5′ OOO 3′, a NLS peptide 5′ VKRKKKP 3′ (SEQ ID NO: 5), capable of binding to Site 2 target DNA can comprise a sequence 5′ JJJTJJJT-OOO-TCCCTCCCTCCGTTCTTTTTCCC-K-OOO-VKRKKKP 3′(SEQ ID NO:6), or a portion or variant thereof that retains the ability to bind to the target DNA.

Aberrant expression of genes is associated with many human diseases such as cancer, Down syndrome, Alzheimer's disease, autoimmune diseases, to name a few. PNA oligomers can be designed based on a portion or full sequence of any known target DNA that controls gene expression as described herein to form a triplex PNA/DNA/PNA, inhibiting expression of the gene.

By “expression” or “gene expression,” it is meant the overall flow of information from a gene (without limitation, a functional genetic unit for producing a gene product, such as RNA or a protein in a cell, or other expression system encoded on a nucleic acid and comprising: a response elements and/or enhancers; an expressed sequence that typically encodes a protein (open-reading frame or ORF) or functional/structural RNA, and a polyadenylation sequence), to produce a gene product (typically a protein, optionally post-translationally modified or a functional/structural RNA). The designated sequence may be all or part of the DNA and may wholly or partially regulate and/or affect the translation or transcription of a gene.

Use of the PNA oligomers to target a DNA site involved in expression of a gene of interest in a cell, will affect gene expression by downregulation or upregulating expression of not only mRNA of the gene of interest and its encoded protein, but also downstream genes that are targets of the gene of interest.

As shown herein below, reduction in C-Myc expression in multiple lymphoma cell lines in vitro and in xenograft mouse models in vivo produced by cellular and nuclear uptake of the novel designed PNA oligomers capable of forming a triplex PNA/DNA/PNA invasion complex with target DNA, resulted in a decrease in tumor cell proliferation and viability exhibiting the superior binding affinity and accumulation in tumors in vivo.

Therefore, in an aspect, a method for increasing or decreasing expression of C-Myc oncogene or associated downstream genes or oncogenes. In one aspect, a method for decreasing gene expression of a C-Myc associated tumor gene comprising downregulating C-Myc expression by providing to a cell a PNA oligomer described herein. In one aspect, the tumor gene is any of MCL1 and/or EZH2 (enhancer of zeste homolog 2), a histone methyltransferase. EZH2 is frequently overexpressed in various malignant tumors including prostate cancer, ovarian cancer, endometrial carcinoma, breast cancer, melanoma as well as hematological malignancies, such as NHL, B-cell lymphoma, and T-cell ALL.

In another aspect, a method for reducing tumor growth in a subject, comprising reducing C-Myc expression in the tumor by administering to the subject a PNA oligomer described herein.

The disclosed compositions can be used for ex vivo or in vivo. The methods typically include contacting a cell with an effective amount of PNA oligomers capable of binding to a target DNA site that controls expression of a desired gene, optionally in combination with a potentiating agent, to modify the expression of a desired gene. As discussed in more detail below, the contacting can occur ex vivo or in vivo. In preferred embodiments, the method includes contacting a population of target cells with an effective amount of the composition, to modify the expression of the desired gene to achieve a therapeutic result.

In one aspect, the PNA oligomers described herein can be used in combination with one or more factor or small molecule, or one or more therapeutic compound that facilitates access to the double-stranded target DNA, or act by different mechanisms to induce death of tumor cells, as presented in Table 2. In the instance where target DNA is genomic DNA, factors that function in opening chromatin structure, such as histone deacetylation inhibitors, including vorinostat, romidepsin, panobinostat, tucidinostat, and belinostat, assist in unwinding of the genomic DNA and can be used in combination with the PNA oligomer. Further, tyrosin kinase inhibitors such as imatinib, EZH2 inhibitors such as 3-deazaneplanocin A (DZNep), epizyme and eisai, tazemetostat, SHR2554, CPI-1205, DS-3201, PF-06821497, and HH2853; isocitrate dehydrogenases (IDHs) inhibitors such as enasidenib, ivosidenib, vorasidenib; BCL-2 inhibitors such as navitoclax, venetoclax; proteasome inhibitors such as bortezomib, carfilzomib, ixazomib; poly(ADP-ribose)polymerases (PARP) inhibitors such as olaparib, rucaparib, niraparib, talazoparib; cytotoxic drug combination; or a combination thereof can be combined with the PNA oligomer. Other therapeutic drugs, e.g., traditional chemotherapy drugs, can also be combined with the PNA oligomer.

TABLE 2
SMALL MOLECULES, FACTORS AND DRUGS
FOR COMBINING WITH PNA OLIGOMER.

| | |
|---|---|
| HDAC | Vorinostat |
| inhibitors | Romidepsin |
| | Belinostat |
| | Panobinostat |
| | Tucidinostat |
| Cytotoxic | CHOP: Cyclophosphamide, Doxorubicin Hydrochloride, |
| drug | Vincristine Sulfate, Prednisone |
| combinations | R-POCH: Rituximab, Cyclophosphamide, Doxorubicin, |
| | Vincristine, Prednisone |
| | EPOCH: Etoposide Phosphate, Prednisone, Vincristine |
| | Sulfate, Cyclophosphamide, Doxorubicin Hydrochloride |
| | COPP: Cyclophosphamide, Vincristine, Procarbazine |
| | Hydrochloride, Prednisone |
| | CVP: Cyclophosphamide, Vincristine sulfate, Prednisone |
| | ICE: Ifosfamide, Carboplatin, Etoposide Phosphate |
| | R-CVP: Rituximab, Cyclophosphamide, Vincristine |
| | sulfate, Prednisone |
| | R-EPOCH: Rituximab, Etoposide Phosphate, Prednisone, |
| | Vincristine Sulfate, Cyclophosphamide, Doxorubicin |
| | Hydrochloride |
| | R-ICE: Rituximab, Ifosfamide, Carboplatin, Etoposide |
| | Phosphate |
| Monotherapies | Selinexor |
| | Venetoclax |
| Poly | Olaparib |
| (ADP-ribose) | Rucaparib |
| polymerase | Niraparib |
| (PARP) | Talazoparib |
| inhibitors | |

For example, the effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder. The molecules can be administered in an effective amount to induce formation of a PNA/DNA/PNA triplex at the DNA target site.

The formulation of the composition comprising the PNA oligomers is made to suit the mode of administration.

Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.).

The disclosed compositions can be administered to or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times each week.

In general, by way of example only, dosage forms useful in the disclosed methods can include doses in the range of about 10² to about 10⁵⁰, or about 10⁵ to about 1040, or about 10¹⁰ to about 10³⁰, or about 10¹² to about 10²⁰ copies of triplex-forming molecules per dose.

The disclosed compositions can be administered directly to a subject for in vivo gene therapy.

The disclosed compositions are preferably employed for therapeutic uses in combination with a suitable pharmaceutical carrier. Such compositions include an effective amount of the composition, and a pharmaceutically acceptable carrier or excipient.

The disclosed compositions of PNA oligomers may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation. Such systems are well known to those skilled in the art and may be optimized for use with the appropriate nucleic acid.

Various methods for nucleic acid or PNA delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994).

Targeting molecules can be proteins, peptides, nucleic acid molecules, saccharides or polysaccharides that bind to a receptor or other molecule on the surface of a targeted cell. The degree of specificity and the avidity of binding to the target cells can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34+ cells, epithelial cells, T cells or any other preferred cell type, as well as receptor and ligands expressed on the preferred cell type. In some embodiments, the moieties target hematopoietic stem cells. The choice of targeting molecule will depend on the method of administration of the particle composition and the cells or tissues to be targeted. The targeting molecule may generally increase the binding affinity of the particles for cell or tissues or may target the particle to a particular tissue in an organ or a particular cell type in a tissue.

The PNA delivery system can be provided to the cell either directly, such as by contacting it with the cell, or indirectly, such as through the action of any biological process. The PNA delivery system can be provided to the cell by endocytosis, receptor targeting, coupling with native or synthetic cell membrane fragments, physical means such as electroporation, combining the PNA delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the PNA delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose, any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro and in vivo. In addition, it has been shown that binding of steroid conjugated oligonucleotides to different lipoproteins in the bloodstream, such as LDL, protect integrity and facilitate biodistribution. Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. These pharmaceutical formulations may be manufactured in a manner that is itself known, e.g., by means of conventional mixing, dissolving, granulating, levigating, emulsifying, encapsulating, entrapping or lyophilizing processes.

In general, methods of administering compounds, including oligonucleotides and related molecules, are well known in the art. In particular, the routes of administration already in use for nucleic acid therapeutics, along with formulations in current use, provide preferred routes of administration and formulation for the PNA oligomers described above.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the PNA oligomer compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes or other nanocarriers.

The compositions may be delivered in a manner which enables tissue-specific uptake of the agent and/or nucleotide delivery system.

Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, poly anhydrides, polycaprolactones, copolyoxalates, polyesteramides, polyorthoesters, polyhydroxybutyric acid, and/or combinations of these. Microcapsules of the foregoing polymers may also be employed. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide-based systems; wax coatings; compressed tablets using conventional binders and excipients; or partially fused implants. Specific examples include erosional systems in which the oligonucleotides are contained in a formulation within a matrix, or diffusional systems in which an active component controls the release rate. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Exemplary subjects include, but are not limited to, mammals such as a human or other primate, a rodent such as a mouse or rat, or an agricultural or domesticated animal such as a dog, cat, cow, horse, pig, or sheep. The subject can be an adult, child, infant, or a multi-cell or single-cell embryo. The methods can include in utero delivery of the composition to an embryo or fetus in need thereof.

The invention is further illustrated by the following non-limiting examples.

### EXAMPLES

### Methods

Synthesis of PNA oligomers: PNAs were synthesized on MBHA (4-methylbenzhydrylamine) resin using Boc chemistry and standard solid phase synthesis protocols. Regular and serine-γPNA-Boc monomers from ASM chemicals and research (Germany) were used. Classical nuclear localization sequence (NLS; VKRKKKP; SEQ ID NO: 5) was conjugated on C terminus using Boc protected amino acids. Boc-MiniPEG3 was used as a linker and carboxytetramethylrhodamine (TAMRA) dye was conjugated on C terminus on γPNA6 and γPNA7-NLS. PNAs were cleaved from the resin in trifluoracetic acid:trifluoromethane sulfonic acid:m-cresol:thioanisole at a ratio of 6:2:1:1 (v/v). Diethyl ether was used to precipitate the PNA followed by washing and vacuum drying. The crude PNAs were then purified via reversed phase high-performance liquid chromatography (RP-HPLC) using 0.1% trifluoroacetic acid (TFA) in acetone and 0.1% TFA in water as mobile phases. Mass spectrometry was used to confirm the molecular weights. The purified PNAs were lyophilized and reconstituted in water and concentration was determined by measuring absorbance at 260 nm via UV-Vis spectrometry.

Gel electrophoresis: The double stranded DNA sequences (dsDNA 1 and dsDNA 2) containing the binding site for γPNA1-NLS and γPNA2-NLS were synthesized via PCR. dsDNA1 was synthesized using below primer sequences with a 27mer complementary region containing binding site for γPNA1-NLS.

dsDNA 1 (101 bp) primers: 5′CTCTGCTTTGGGAACCCGGGAGGGGCGCTTATGGGGAGGGTGGGGAGGGTGGG GAAGGTGGGGA 3′ (SEQ ID NO:12) 5′AGAGTGCTCGGCTGCCCGGCTGATGTCTCTTCCCCACTCCCCACCTTCCCCACC CTCCCCACCC 3′ (SEQ ID NO:13) dsDNA 2 was synthesized using below primers with 23mer complementary region containing the binding site for γPNA2-NLS. dsDNA (101 bp) primers: 5′TCCTGCCTCGAGAAGGGCAGGGCTTCTCAGAGGCTTGGCGGGAAAAAGAACGG AGGGAGGGA 3′ (SEQ ID NO:14) 5′AGATAAAGCCCCGAAAACCGGCTTTTATACTCAGCGCGATCCCTCCCTCCGTTC TTTTTCCC 3′ (SEQ ID NO: 15) 10×PCR reaction buffer (5 μl), 50 mM MgCl₂ (1.5 μl), 10 mM dNTP (1 μl), 10 μM primers (1 μl each primer), and DNA Taq polymerase (0.5 μl) were mixed with water up to a volume of 50 μl. DNA was amplified in a thermal cycler using the conditions: 95° C. (2 min), 95° C. (30 sec), 55° C. (30 sec), 72° C. (1 min), 72° C. (10 sec) for a total of 10 cycles. The PCR reaction mixtures were pooled and quenched using 0.2× volume of 10 mM EDTA followed by dsDNA extraction with 1× chloroform: phenol: isoamyl alcohol (24:25:1) twice. The aqueous fractions were collected, combined and precipitated by adding 1 μl glycogen, 0.1×3 M sodium acetate, and 3× absolute ethanol at −20° C. for 35-40 mins. The precipitated dsDNA was collected by centrifugation at 15000 RPM for 5 mins, the pellet was washed with 70% ethanol twice, air-dried and reconstituted in DNAse free water. The concentration was measured using Nanodrop™. The purified dsDNA 1 and dsDNA 2 were then incubated with different concentrations of γPNA1-NLS and γPNA2-NLS respectively in 10 mM sodium phosphate buffer at 37° C. for 17 hours. Samples were then separated on 10% polyacrylamide gel at 120V for 40 mins. The bound and unbound fraction of dsDNA was visualized using SYBR™ gold staining and Gel Doc™ EZ imager (Bio-Rad, USA).

Cellular uptake: HeLa and U2932 cells were purchased from ATCC (USA), cultured in EMEM and RPMI media, respectively (Invitrogen, USA), supplemented with 10 FBS and 1% Penstrep at 37° C. and 5% CO₂.

Confocal Microscopy: HeLa cells were seeded in 8 chamber slides at 50,000 cells/well. U2932 cells were seeded in 24 well plate at 100,000 cells per well. γPNA7-NLS containing TAMRA was added to the cells at 2 μM concentration. After 24 h, cells were washed with PBS to remove uninternalized PNA. The nucleus of live cells was stained using Hoechst dye and were visualized under confocal microscope (Nikon AIR, USA) while cells are maintained in CO₂ independent media. Z-stacks were captured using 2 μm step size and maximum intensity projection images were obtained using imageJ software. Flow cytometry: U2932 cells treated with different γPNA7-NLS concentrations (1, 2, 4, and 8 μM) for 24 h were collected and washed with PBS. Cells were then suspended in PBS and analyzed using LSR Fortessa X-20 cell analyzer (BD Biosciences, USA). The data was analyzed using Flowjo software.

Cell viability: U2932 and Raji cells were seeded in 96 well plates at 20,000 cells/well. PNAs were added to the media at different concentrations (1, 2, 4, and 8 μM). After 24 h, the dead cells were stained with the trypan blue dye and count was measured using the cell counter (Bio-Rad, USA). For testing the combination of PNAs with HDAC inhibitors, U2932 cells were pre-treated with the respective inhibitors (valproic acid at 2 mM, vorinostat at 2.5 μM, romidepsin at 10 nM) for 24 h followed by addition of PNAs at 8 μM. The cell viability was then measured via trypan blue staining on day 1, 2, and 4.

Gene expression: The levels of mRNA were measured in different cell lines using real time-polymerase chain reaction (RT-PCR). Both untreated (PBMC, U2932, Raji, Daudi) and PNA treated lymphoma cells were collected at the end of treatment. Total RNA was extracted using Qiagen RNeasy® kit (USA) and concentration was measured by Nanodrop™. cDNA was synthesized using high capacity cDNA reverse transcription kit (Applied Biosystem, USA) following the recommended cycling conditions. Taqman® gene expression assay for C-Myc, EZH2, BCL2, MCL1 and GAPDH were used to amplify the respective mRNAs using the specified cycling conditions in the CFX Real-Time PCR detection system. GAPDH was used as the reference gene and fold change in mRNA expression was obtained by normalizing against the untreated cells or cells treated with HDAC inhibitors.

In vivo studies in xenograft mice model: U2932 derived xenografts were generated by implanting 10 million cells subcutaneously on the lower right flank of the female NSG mice (NOD.Cg-Prkdc^{scid}Il2rg^{tm1Wj1}/SzJ, #005557, Jackson Laboratories, USA) of 5-6 weeks. The mice were maintained in the animal care facility at the University of Connecticut following the required Institutional Animal Care and Use Committee guidelines (IACUC). Mice developed palpable tumors after 3-4 weeks of implantation. Tumor dimensions including length (l), breadth (b), and height (h) were measured using a caliper and volume was calculated using the formula for ellipsoid (0.5236×lbh).

Biodistribution studies: Mice with 600-800 mm³ tumor volume were used for the biodistribution studies. γPNA7-NLS, γPNA6 and regular PNA-TAMRA (23 mer with 3 arginine residues) was injected systemically (retro-orbital) at 5 mg/kg dose. Live imaging of animals was performed using in vivo imaging (IVIS®) spectrum CT and epifluorescence was recorded at excitation/emission wavelength of 549/578 nm at 0, 0.25, 1, 2, 4, 6, 8 and 24 h. Mice were euthanized after 6 h and 24 h. Organs were collected and imaged via IVIS® to determine organ localization of PNA. Further organs were frozen in optimum cutting temperature media (OCT) at −80° C. The tumor, liver, and kidney from treated and untreated mice were sectioned using a cryostat to obtain 10 μm tissue sections. The sections were washed in PBS followed by fixation in 10% neutral buffered formalin. After washing with PBS, sections were permeabilized using 0.2% Triton™-X. The nucleus was stained using mounting media with DAPI (Invitrogen, USA). Sections were allowed to harden overnight, and images were taken using 60× oil lens on a Nikon AIR confocal microscope.

Survival study: Mice bearing tumors about 100-150 mm³ were divided into 4 groups. Mice were treated with either γPNA2-NLS, γPNA3, Scr-γPNA4-NLS, or saline. PNAs were administered at 5 mg/kg dose on day 1, 4, 7, and 10. The change in tumor volume was measured every day. Mice were euthanized when the tumor volume reached 2000 mm³. Blood was collected via cardiac punter in 1.5 ml tubes containing 0.5 M EDTA. Organs including tumor, liver, kidney, spleen, heart and lung were collected. Tumor fraction and all organs were kept in 10% formalin and submitted for histology. The complete blood count analysis was performed on the collected blood samples using Sysmex CBC analyzer. Plasma was separated from the blood samples and submitted to Antech diagnostic for blood chemistry analysis to quantify the levels of alanine transaminase, lactate dehydrogenase, aspartate transaminase and blood urea nitrogen.

In vivo studies in transgenic mice model: Ep-myc mice model of Burkitt lymphoma (B6.Cg-Tg(IgHMyc)22Bri/J, #002728, Jackson laboratories) which overexpresses c-myc was also used to test the efficacy of the designed PNA-NLS.

Biodistribution study: γPNA7-NLS containing TAMRA was administered via retro-orbital and subcutaneous route at 5 mg/kg dose in mice with visibly enlarged lymph nodes (cervical, brachial, axillary, and inguinal). Mice were euthanized after 24 h and organs were collected followed by imaging via IVIS®. All major organs and enlarged lymph nodes were collected and frozen in OCT media. The lymph nodes and organs were sectioned at 10 μm thickness using cryostat. The sections were then fixed, permeabilized and nucleus was stained with DAPI. The localization of PNA in lymph nodes was studied via confocal microscopy. Images were taken using 60× oil lens on a AIR confocal microscope.

Efficacy study: Ep-myc mice (male and female) with visibly enlarged lymph nodes were divided into two groups. Mice were treated with γPNA5-NLS subcutaneously at 60 mg/kg dose over 2 days. Mice were euthanized on day 3 followed by collection of major organs and lymph nodes. Organs and sections of lymph nodes were fixed in formalin for histology. Lymph nodes were further processed into single cell suspension by mashing them and passing through the 40 μm filter in RPMI media. Cells were suspended in RBC lysis buffer for 2 mins followed by washing with PBS. Single cell suspension was then collected for protein analysis.

Western blot analysis: Total protein was extracted from collected lymphocytes using 1×RIPA buffer (Cell signaling technology, USA) containing protease inhibitor. The concentration of protein was determined using absorbance-based DC protein assay (Bio-Rad, USA). Equal amount of protein (25-40 μg) was separated on 4-20% SDS-PAGE gel followed by transfer to the PVDF membrane. C-Myc protein was probed using rabbit monoclonal primary antibody (Cell signaling technology, #5605) at 1:500 dilution in 3% BSA at 4° C. overnight. Cyclophilin B (#43603) and vinculin (#13901) was also probed as the endogenous control using rabbit monoclonal antibodies. The bands were detected using anti-rabbit IgG HRP linked secondary antibody (Cell signaling technology, #7074) (1:2000 dilution, 5% milk in TBST) and HRP substrate (Millipore sigma). The blots were imaged using ChemiDoc imager (Bio-Rad, USA) and band intensities were quantified using imageJ.

### Example 1: Design and Synthesis of PNAs

C-Myc is a transcription factor which regulates 10-15% of human genes by forming a complex with myc associated factor-X (MAX) and recruiting co-activators or repressors. It belongs to the family of “super-transcription” factor which activates the transcription by interacting with E box DNA sequences (CACGTG) located in the regulatory regions of the target genes. C-Myc regulates multiple cellular process including growth, division, DNA replication, metabolism, and protein synthesis. The overexpression of C-Myc was first identified as target of t(8;14) q(24;32) chromosomal translocation in Burkitt's Lymphoma, where it is juxtaposed to immunoglobin heavy chain (IGH) locus on chromosome 14. In addition, the translocation, gene rearrangement, amplification and mutation also induce the overexpression of c-myc protein in lymphoid neoplasms. C-Myc has been reported to play a role in maintaining cancer stem cell like properties which leads to metastasis, recurrence and chemoresistance. The elevated levels of C-Myc have been reported in cisplatin resistant cancer cells. Further studies in transgenic mice have shown that inhibition of oncogenic C-Myc induces tumor regression, highlighting it as a therapeutic target for treatment of C-Myc driven tumors.

The overexpression of C-Myc (>70%) is also reported in a majority of Diffuse Large B-Cell Lymphoma (DLBCL) cases due to the genetic alterations introduced via germinal center somatic mutational machinery. Diffuse Large B-Cell Lymphoma (DLBCL) is an aggressive lymphoma that can arise in lymph nodes or outside of the lymphatic system, in the gastrointestinal tract, testes, thyroid, skin, breast, bone or brain. A combination of chemotherapy and a monoclonal antibody targeting CD20 remains the backbone of most treatments. The most widely used treatment for DLBCL is R CHOP (rituximab, cyclophosphamide, doxorubicin, vincristine, and prednisone) that is usually given in 21-day cycles.

The C-Myc oncogene, located on chromosome 8, consists of three exons. Exon 1 is non-coding while exon 2 and exon 3 encodes for the c-myc protein. There are four transcriptional promoters reported for regulating the expression of c-myc. Exon 1 contains two promoter regions (P1 and P2) which regulates the transcription initiation. We selected targets sites containing homopurine region upstream of both P1 and P2. Next, we designed the complementary PNA sequence to the respective target sites (FIG. 1).

PNAs were synthesized using boc chemistry and standard protocols for solid phase synthesis. NLS peptide was conjugated on the C terminus of PNAs. We also used modified gamma modified PNA monomers at alternate position during the synthesis of tcPNAs. In addition, 5-carboxy-tetramethylrhodamine dye (TAMRA) was conjugated on the C terminus of the PNA (γPNA6 and γPNA7-NLS) with polyethylene glycol as linker. TAMRA dye conjugated PNAs with and without NLS will help to establish the role of NLS in cellular, nuclear and in vivo delivery of PNAs. We also synthesized a scramble sequence of γtcPNA containing NLS (γScr-PNA4-NLS) to use as control in in vitro as well as in vivo studies. In order to test the efficacy in the transgenic mice model of burkitt lymphoma, we will also design a tcPNA sequence (γPNA6-NLS) targeting the promoter region 2 of the mouse C-Myc oncogene.

### Example 2: Biophysical Characterization of PNAs

After the completion of synthesis, PNAs were purified by reverse phase high performance liquid chromatography (RP-HPLC). The molecular weight of purified PNAs was confirmed via mass spectrometry. Further, we studied the in vitro binding of the synthesized γPNA1-NLS, γPNA2-NLS, and Scr-γPNA4-NLS with the double stranded DNA (dsDNA) containing the target site via gel electrophoresis as previously reported. Here, we first generated 101 base pair dsDNA1 containing the target site for γPNA1-NLS and 101 bp dsDNA 2 containing the target site for γPNA2-NLS using standard PCR conditions followed by purification. Next, 1 M of purified dsDNA was incubated with the γPNA-NLS at different ratios (1:0.1, 1:0.2, 1:0.4, 1:0.8, and 1:1) in 10 mM sodium phosphate buffer at 37° C. for 17 hours. Samples were then separated on 10% polyacrylamide gel followed by staining with SYBR gold to visualize the bound and unbound fraction of the dsDNA. We observed binding of the γPNA1-NLS and γPNA2-NLS with the dsDNA1 and dsDNA2 respectively even at 1:0.1 ratio, while complete binding was observed at 1:0.4 ratio indicating superior binding affinity and invasion capabilities of the designed γPNA-NLS towards the dsDNA.

### Example 3: γPNA-NLS Exhibits Superior Cellular Uptake In Vitro

Next, we studied the cellular uptake of γPNA7-NLS in HeLa and diffuse large B cell lymphoma (U2932) cells. We incubated HeLa or U2932 cells with γPNA7-NLS (2 M) for 24 h and studied the cellular uptake in live cells by confocal and flow cytometry-based assays. HeLa cells showed significant accumulation of γPNA7-NLS in the cytoplasm (FIG. 3A). Further we also observed accumulation of γPNA7-NLS within the nucleus where TAMRA fluorescence overlaps with the Hoechst dye staining the nucleus. Similarly, we observed accumulation of γPNA7-NLS in both the nucleus and cytoplasm of U2932 cells (FIG. 3B). Moreover, these results were confirmed by quantifying the uptake of γPNA7-NLS in U2932 cells in dose dependent manner via flow cytometry (FIG. 3C). After 24 h of treatment with γPNA7-NLS, we observed a dose dependent increase in cellular uptake with significant accumulation observed at a dose of 8 M.

### Example 4: γPNA-NLS Efficiently Inhibits C-Myc in Multiple Lymphoma Cell Lines

We tested and compared the efficacy of γPNA-NLS targeting selected regions in both burkitt lymphoma (Raji and Daudi) and DLBCL (U2932) cells. We first compared the levels of C-Myc oncogene in Raji, Daudi, and U2932 cells against normal peripheral blood mononuclear cells (PBMC). We observed approximately 2.7-fold increase in the levels of C-Myc in Raji cells, while U2932 and Daudi cells showed approximately 1.5-fold upregulation of C-Myc oncogene (FIG. 4A). Next, we tested the impact of γPNA1 NLS, γPNA2-NLS, γPNA3, and ScrγPNA4-NLS on viability of U2932 and Raji cells at different doses. We noted a significant decrease in the viability of both U2923 and Raji cells after treatment with γPNA2-NLS in comparison to γPNA1-NLS and γPNA3 after 24 h (FIGS. 4B and 4C). However, ScrγPNA4-NLS showed no impact on the viability of both U2932 and Raji cells. Further, we quantified the C-Myc oncogene levels via RT-PCR after treatment with designed PNAs in U2932 and Raji cells. We observed >50% decrease in the levels of C-Myc in both U2932 and Raji cells after treatment with γPNA2-NLS. However, we did not observe any change in levels of C-Myc after treatment with γPNA1-NLS in either of the cell lines (FIGS. 4D and 4E). These results are consistent with the reports that promoter 2 regulates more than 80% of the C-Myc transcription. We also tested the levels of downstream targets of C-Myc including MCL1, BCL2, and enhancer zester homolog 2 (EZH2). MCL1 and BCL2 are anti-apoptotic proteins which allow continuous proliferation of lymphoma cells by preventing the apoptosis. EZH2 is a histone-lysine-N-methyl transferase enzyme which cause methylation of histone protein, leading to compaction of chromatin structure and inhibiting the overall transcription process. We observed a significant decrease in the levels of MCL1 and EZH2 in U2932 While Raji cells also showed downregulation of BCL2 and EZH2 after treatment with γPNA2-NLS, while no change in their levels was observed for γPNA1-NLS and Scr-γPNA4-NLS consistent with the C-Myc expression results. We also tested the knockdown of C-Myc at different doses of γPNA2-NLS in U2932 cells using ScrγPNA4-NLS as control (FIG. 4F), where γPNA2-NLS showed maximum knockdown at 8 M while ScrγPNA4-NLS showed no activity.

### Example 5: γPNA-NLS Exhibits Superior Accumulation in Tumors in Vivo

Next, we tested the biodistribution of γPNA7-NLS containing a rhodamine (TAMRA) dye in a xenograft mice model of lymphoma. First, we developed xenograft mice by implanting 1×10⁷ U2932 cells subcutaneously in nod-scid gamma mice (NSG mice). Mice bearing tumors approximately 600-800 mm³ were used for the biodistribution study. We compared the biodistribution of γPNA7-NLS against only PNA-TAMRA without NLS peptide to establish the role of NLS in superior pharmacokinetic properties and tumor accumulation. We retro-orbitally injected the γPNA7-NLS and PNA-TAMRA in xenograft mice at the dose of 5 mg/kg. We performed live imaging of the mice at different time points including 0, 0.25, 1, 2, 4, and 6 h after systemic administration using IVIS imaging. Initially, we noted rapid distribution of both the PNAs across the body and passive accumulation in the tumors. However, PNA-TAMRA completely eliminated from the body by 4 h, while γPNA7-NLS showed tumor accumulation until 6 h (FIG. 5A). We euthanized the mice after 6 h of systemic administration and harvested the organs. When imaged via IVIS, we observed significant TAMRA intensity from tumors of γPNA7-NLS treated mice, while PNA-TAMRA showed minimal signal (FIG. 5B). We also quantified the fluorescence intensity from the organs harvested both γPNA7-NLS and PNA-TAMRA treated mice after 6 h. We observed more than 6-fold higher accumulation of γPNA7-NLS in tumors in comparison to PNA-TAMRA (FIG. 5C). Further, the accumulation of γPNA7-NLS in tumor was higher in comparison to the other organs including liver, kidney, and intestine. Confocal microscopy imaging revealed the superior accumulation of γPNA7-NLS in tumor in comparison to PNA-TAMRA which showed no tumor delivery. Unlike γPNA7-NLS, PNA-TAMRA was majorly present in kidney (FIG. 5D). The results from this study established the superior pharmacokinetic properties and tumor accumulation of γPNA7-NLS in comparison to PNA-TAMRA. Hence NLS not only assists in cellular and nuclear delivery of PNA, but also results in higher accumulation and retention in the tumors in vivo.

Next, we tested the biodistribution of γPNA7-NLS and γPNA6 with same PNA sequence except for NLS peptide in xenograft mice until 24 h of systemic administration at a dose of 5 mg/kg. We performed live imaging of treated xenograft mice at different time points including 0, 0.25, 2, 4, 8, and 24 h after systemic injection of γPNA7-NLS and γPNA6. We noted significant accumulation of γPNA7-NLS in tumors starting from 0.25 h, which retained within the tumors until 24 h (FIG. 6A). We euthanized the mice and collected the tumors as well as other organs to study the distribution of PNA at organ level via IVIS imaging. We observed higher fluorescence signal in γPNA7-NLS group than γPNA6 after 24 h of systemic administration of γPNA7-NLS indicating longer retention of γPNA7-NLS in tumors (FIG. 6B). We also investigated the accumulation of γPNA7-NLS and γPNA6 in tumors, liver and kidney via confocal imaging. The harvested tumors were frozen in optimum cutting temperature medium and 10 m sections were made using a cryostat followed by processing to stain the nucleus. γPNA7-NLS showed significantly higher accumulation in tumors after 24 h than γPNA6 (FIG. 6C).

### Example 6: γPNA-NLS Inhibits Tumor Growth in a Xenograft Mice Model of Lymphoma

To evaluate the impact on tumor growth, we established U2932 cell line derived xenograft mice model. We compared the efficacy of γPNA2-NLS and γPNA3 after systemic administration in U2932 xenograft mice. Saline treated mice were used as control and ScrγPNA4-NLS was used as negative control. Once the tumor volume reached 100-150 mm³, PNAs were administered systemically on day 1, 4, 7, and 10 and tumor growth was monitored by measuring the tumor dimensions (length, breadth and height) using a vernier caliper every day. We observed a significantly reduced tumor growth in both γPNA2-NLS and γPNA3 treated mice in comparison to the control groups (FIG. 7A). However, γPNA2-NLS showed superior efficacy in comparison to the γPNA3 treated mice.

In comparison to the saline (21 days) and scr-γPNA4-NLS (19.5 days) treated group, γPNA2-NLS significantly increased the survival of the mice to 29 days while γPNA3 only improved the survival to 23 days (FIG. 7B). We did not notice any change in the blood profile of treated mice in comparison to the control group (FIG. 7C). Similarly, blood chemistry panel including alanine transaminase (ALT), aspartate transaminase (AST), lactate dehydrogenase (LDH), and blood urea nitrogen (BUN) showed no significant change among different treated groups in comparison to the saline treated mice (FIG. 7C). These results indicated the safety of systemically administered PNAs after multiple dosing.

### Example 7: γPNA-NLS Inhibits C-myc in a Transgenic Mice Model of Lymphoma

Simultaneously, we also tested the efficacy of γPNA-NLS targeting the mouse C-Myc oncogene (γPNA5-NLS) in the transgenic mice model (Ep-myc) of Burkitt lymphoma. These mice develop lymphomas within 10 to 16 weeks of age. First, we studied the biodistribution of γPNA-NLS TAMRA in transgenic mice after intravenous (IV) and subcutaneous (SC) route of administration as target site of accumulation is enlarged lymph nodes. After 24 hours of IV and SC administration of γPNA-NLS TAMRA (5 mg/kg), SC route showed significantly higher accumulation of γPNA-NLS TAMRA in lymph nodes than IV route (FIG. 8A) via IVIS imaging. These results were further supported by the confocal microscopy images of the lymph node sections where SC administered γPNA-NLS TAMRA accumulated significantly in multiple lymph nodes (cervical and brachial) while IV administration resulted in minimal accumulation in the lymph nodes (FIG. 8B). Next, we administered the γPNA5-NLS in the transgenic mice via SC route at a dose of 60 mg/kg over 2 days and euthanized the mice on day 3 to evaluate the change in c-myc levels. We observed a substantial decrease in c-myc protein levels via western blot analysis in both brachial and axillary lymph nodes of γPNA5-NLS treated mice in comparison to the untreated group (n=4 mice per group) (FIG. 8C).

### Example 8: Combination Therapy of γPNA-NLS with Romidepsin

Next, we evaluated the efficacy of γPNA-NLS with an FDA approved histone deacetylation inhibitor (HDACi), Romidepsin. HDACi act by inhibiting the deacetylation of histone leading to the more open and accessible chromatin structure. This may allow the γPNA-NLS to have better accessibility at the target site. Hence, we tested both γPNA1-NLS and γPNA2-NLS in combination with romidepsin in U2932 cells. We pretreated the U2932 cells with romidepsin for 24 h and added γPNA1-NLS and γPNA2-NLS at 8 uM dose. We evaluated the viability of U2932 cells over a period of 4 days. Romidepsin alone showed a 60% decrease in viability by day 4, however combination of romidepsin with both γPNA1-NLS and γPNA2-NLS showed an approximately 80% decrease in U2932 cell viability (FIGS. 9A and 9B). Hence, the combination of romidepsin with γPNA1-NLS and γPNA2-NLS showed synergistic activity. We further evaluated the change in C-Myc and BCL-2 levels at day 4 in combination treatment of romidepsin with γPNA1-NLS or γPNA2-NLS and compared with only romidepsin treated U2932 cells as control. We noted an approximately 50% decrease in C-Myc levels after combining romidepsin with γPNA1-NLS, while γPNA2-NLS showed >90% decrease in C-Myc levels on combination with romidepsin (FIG. 9C). Similar results were observed for BCL-2 levels, where combination treatment of γPNA1-NLS or γPNA2-NLS with romidepsin showed reduced levels in comparison to only romidepsin (FIG. 9D).

We also tested the combination of PNA2-NLS with vorinotsat, another HDACi, which also showed synergistic activity in U2932 cells (FIG. 10A). Further, valproic acid has been reported in literature to also increase the accessibility to the genomic DNA. The combination of PNA2-NLS with valproic acid also showed significant decrease in viability of U2932 cells in comparison to the individual treatments (FIG. 10B). The gene expression results supported the viability results, where higher knockdown of c-myc was observed for combination of PNA2-NLS with vorinostat and valproic acid (FIGS. 10C and 10D). Alternative to the HDACi, we also tested the synergistic activity of PNA2-NLS with venetoclax, a bcl-2 inhibitor, and CHOP therapy, which are FDA approved for the treatment of lymphomas and several other tumors. We noted significant reduction in viability of U2932 cells when treated with the combination of PNA2-NLS and venetoclax or PNA2-NLS and CHOP over time (FIGS. 10E and 10F).

In conclusion, targeting genomic DNA with the PNA based platform described herein to inhibit transcription of genes involved in disease provides a superior strategy for targeting proteins associated with malignant and non-malignant disorders, is easily scalable and clinically translatable.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A peptide nucleic acid analogue (PNA) oligomer that forms a PNA/DNA/PNA triplex invasion complex with a homopurine region of a target deoxyribonucleic acid (DNA), wherein the PNA oligomer has the formula:
5′-first PNA segment-flexible linker 1-second PNA segment-3′
wherein
the first PNA segment is complementary to a homopurine stretch in the target DNA,
the second PNA segment is complementary to a region of the target DNA including the homopurine stretch,
a nuclear localization signal (NLS) peptide is conjugated to a carboxyl-terminus (C-terminus), an amino-terminus (N-terminus), or both the C-terminus and N-terminus of the PNA oligomer,
the first PNA segment and the second PNA segment form the PNA/DNA/PNA triplex structure with the target DNA, and
the target DNA is genomic DNA.

2. The PNA oligomer of claim 1, wherein the first PNA segment, the second PNA segment, or both the first PNA segment and the second PNA segment comprises one or more gamma-modified monomer units.

3. The PNA oligomer of claim 1, wherein the first PNA segment comprises 3-10 pyrimidines.

4. The PNA oligomer of claim 1, wherein the first PNA segment comprises one or more pseudoisocytosine units.

5. The PNA oligomer of claim 1, wherein the first PNA segment comprises only pseudoisocytosine units and thymidine units.

6. The PNA oligomer of claim 1, wherein the flexible linker comprises 1-10 units of 8-amino-3,6-dioxaoctanoic acid, 8-amino-2, 6,10-trioxaoctanoic acid, or 11-amino-3, 6, 9-trioxaundecanoic acid.

7. The PNA oligomer of claim 1, wherein the NLS comprises the amino acid sequence 5′-VKRKKKP-3′.

8. The PNA oligomer of claim 7, wherein the NLS is conjugated to the PNA oligomer via a lysine amino acid.

9. The PNA oligomer of claim 8, wherein the target DNA is in a transcriptional control region of a gene.

10. The PNA oligomer of claim 9, wherein the transcriptional control region is a promoter, an enhancer, or a transcription-factor binding region of a gene.

11. The PNA oligomer of claim 10, wherein the gene is overexpressed in a disease state.

12. The PNA oligomer of claim 11, wherein the gene is an oncogene.

13. The PNA oligomer of claim 12, wherein the oncogene is Cellular myelocytomatosis (C-myc) and the second PNA segment is complementary to a region of C-myc and has the sequence 5′-TCCCTCCCTCCGTTCTTTTTCCC-3′ (SEQ ID NO: 16).

14. The PNA oligomer of claim 1, further comprising a detectable label.

15. A composition comprising the PNA oligomer of claim 1 and a pharmaceutically acceptable excipient.

16. A method for inhibiting gene transcription of a target genomic DNA involved in health disorders in a subject, the method comprising
providing to a cell of the subject in vivo or ex vivo a PNA oligomer according to claim 1, wherein the binding of the PNA oligomer to the targeted DNA reduces expression of the targeted gene.

17. The method of claim 16, further comprising administering a factor that opens chromatin DNA structure, wherein administering makes the target DNA accessible for PNA oligomer invasion.

18. The method of claim 17, wherein the factor is a histone deacetylation inhibitor (HDACi), a cytotoxic drug combination, a monotherapy, a poly (ADP-ribose) polymerase (PARP) inhibitor, or a combination thereof, wherein
the HDACi is vorinostat, romidepsin, belinostat, panobinostat, tucidinostat, or a combination thereof;
the monotherapy is selinexor, venetoclax, or a combination thereof;
the poly (ADP-ribose) polymerase (PARP) inhibitor is Olaparib, rucaparib, niraparib, talazoparib, or a combination thereof; and
the cytotoxic drug combination is:
cyclophosphamide, doxorubicin hydrochloride, vincristine sulfate, and prednisone (CHOP);
rituximab, cyclophosphamide, doxorubicin, vincristine, and prednisone (R-POCH);
etoposide phosphate, prednisone, vincristine sulfate, cyclophosphamide, doxorubicin hydrochloride (EPOCH);
cyclophosphamide, vincristine, procarbazine hydrochloride, and prednisone (COPP);
cyclophosphamide, vincristine sulfate, and prednisone (CVP);
ifosfamide, carboplatin, and etoposide phosphate (ICE);
rituximab, cyclophosphamide, vincristine sulfate, and prednisone (R-CVP);
rituximab, etoposide phosphate, prednisone, vincristine sulfate, cyclophosphamide, and doxorubicin hydrochloride (R-EPOCH);
rituximab, ifosfamide, carboplatin, and etoposide phosphate (R-ICE); or
a combination thereof.

19. The method of claim 18, wherein the factor is administered prior to, along with, or post administration of the PNA oligomer.

20. A method for increasing survival, reducing effects of the cancer, or reducing the size of a tumor, in a subject with Burkitt's lymphoma or diffuse large B cell lymphoma or another cancer overexpressing the oncogene, the method comprising administering to said subject a PNA oligomer of claim 13, and optionally, administering a factor that opens chromatin DNA, wherein administering the factor makes the target DNA accessible for PNA oligomer invasion.


SEQUENCE LISTING (16 sequences)

SEQ ID NO: 1 - DNA, 3588 bp, Homo sapiens
  source 1..3588: mol_type = genomic DNA
cccctttcccccgaattgttttctcttttggaggtggtggagggagagaaaagtttacttaaaatgcctttgggtgagggaccaaggatgagaagaatgttttttgtttttcatgccgtggaataacacaaaataaaaaatcccgagggaatatacattatatattaaatatagatcatttcagggagcaaacaaatcatgtgtggggctgggcaactagctnaagtcgaagcgtaaataaaatgtgaatacacgtttgcgggttacatacagtgcactttcactagtattcagaaaaaattgtgagtcagtgaactaggaaattaatgcctggaaggcagccaaattttaattagctcaagactnccccccccccccaaaaaaaggcacggaagtaatactcctctcctcttctttgatcagaatcgatgcattttttgtgcatgaccgcatttccaataataaaaggggaaagaggacctggaaaggaattaaacgtccggtttgtccggggaggaaagagttaacggtttttttcacaagggtctctgctgactcccccggctcggtccacaagctctccacttgccccttttaggaagtccggtcccgcggttcgggtaccccctgcccctcccatattctcccgtctagcacctttgatttctcccaaacccggcagcccgagactgttgcaaaccggcgccacagggcgcaaaggggatttgtctcttctgaaacctggctgagaaattgggaactccgtgtgggaggcgtgggggtgggacggtggggtacagactggcagagagcaggcaacctccctctcgccctagcccagctctggaacaggcagacacatctcagggctaaacagacgcctcccgcacggggccccacggaagcctgagcaggcggggcaggagggccggtatctgctgctttggcagcaaattgggggactcagtctgggtggaaggtatccaatccagatagctgtgcatacataatgcataatacatgactccccccaacaaatgcaatgggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgtgggcgtcctgggaagggagatccggagcgaatagggggcttcgcctctggcccagccctcccgctgatcccccaaccagcggtccgcaacccttgtcgcatccacgaaactttgtccatagcagcgggcggacactttgcactggaacttacaacacccgagcaaagacgcgactctcccgacgcggggaggctattctgcccatttggggacacttccccgccgctgccaggacccgcttctctgaaaggctctccttgcagctgcttagacgctggatttttttcgggtggaaaaccaggtaagcaccgaagtccacttgccttttaatttatttttttatcactttaatgctgagatgagtcgaatgcctaaatagggtgtcttttctcccattcctgcgctattgacacttttctcagagtagttatggtaattggggctggggtggggggtaatccagaactggatcggggtaaagtgacttgtcaagatggcagaggagaaggcagagggaaaacgggaatggtttttaagactaccctttcgagatttctgccttatgaatatattcacgctgactcccggccggtcggacattcctgctttattgtgttaattgctctctgcgttttggggggctgggggttgctttgcggtgggcagaaagccccttgcatcctgagctccttggagtagggaccgcatatcgcctgtgtgagccagatcgctccgcagccgctgacttgtccccgtctccgggagggcatttaaatttcggctcaccgcatttctgacagccggagacggacactgcggcgcgtcccgcccgcctgtccccgcggcgattgcaacccgccctgatccttttaagaagttggcatttggctttttaaaaaacaataatacaatttaaaacctgggtctctagaggtgttaggacgtggtgttgggtaggcgggtacctagaacagacccaattacacggttgaataaatgcagtcggtgtgctcaggtgctgagatccatggccagaaagagtctggtgatagatttatgcccagaggagtttgtacctgcctggttgcttgagggttaaaatgggttaaaatataggatgatgactcagagctggagacaaccttagcattcgctttccagatgaagcccctggagaagacgcaatcttcctgagatccctctactggcttttggctgaattagcttatgacctcagggccctggttgcatcctaactgattatcaagcaagtaagacctcaggtcttctgagatgcaggatgcttacttgccttactactaagatgaagctttcaaactcaccccgtttccaagtccatgccaggtcttatgcccacacccagactggcccagtcaacaaatatttttgatgaagcttaacagtgatggcagccacagcaagggggacgggatccctgatcgcttctcaggctccagctctggggctgagcgctacctcaccatctccagcctccagtctgaggatgaggctgactattactgtcagacctggggcactggcattcaagggatgttcggcggagggaccaagctgaccgtcctaggtgagtctcttctcccctctccttccccgctctcgggacattctgctgttt

SEQ ID NO: 2 - DNA, 23 bp, Homo sapiens
  source 1..23: mol_type = genomic DNA
gagggtggggagggtggggaagg

SEQ ID NO: 3 - DNA, 23 bp, Homo sapiens
  source 1..23: mol_type = genomic DNA
gggaaaaagaacggagggaggga

SEQ ID NO: 4 - DNA, 23 bp, Synthetic construct
  source 1..23: mol_type = other DNA
  misc_feature 1: note = JJJJTTJJ-linker- is covalently linked to the backbone, wherein J is pseudoisocytosine and the linker is 11-Amino-3,6,9-Trioxaundecanoic Acid (DCHA), represented as -OOO-
  misc_feature 23: note = -K-linker-SEQ ID NO: 5 is covalently linked to the backbone, wherein the linker is 11-Amino-3,6,9-Trioxaundecanoic Acid (DCHA), represented as -OOO-
  modified_base 1..23: mod_base = OTHER; note = peptide nucleic acid
ccttccccaccctccccaccctc

SEQ ID NO: 5 - protein, 7 aa, Synthetic construct
  source 1..7: note = nuclear localization signal
VKRKKKP

SEQ ID NO: 6 - DNA, 23 bp, Synthetic construct
  source 1..23: mol_type = other DNA; note = gammaPNA to target C-Myc Exon 1
  misc_feature 1: note = JJJTJJJT-linker- is covalently linked to the backbone, wherein J is pseudoisocytosine and the linker is 11-Amino-3,6,9-Trioxaundecanoic Acid (DCHA), represented as -OOO-
  misc_feature 23: note = -K-linker-SEQ ID NO: 5 is covalently linked to the backbone, wherein the linker is 11-Amino-3,6,9-Trioxaundecanoic Acid (DCHA), represented as -OOO-
  modified_base 1..23: mod_base = OTHER; note = peptide nucleic acid
tccctccctccgttctttttccc

SEQ ID NO: 7 - DNA, 23 bp, Synthetic construct
  source 1..23: mol_type = other DNA; note = gammaPNA
  misc_feature 1: note = JJJTJJJT-linker- is covalently linked to the backbone, wherein J is pseudoisocytosine and the linker is 11-Amino-3,6,9-Trioxaundecanoic Acid (DCHA), represented as -OOO-
  misc_feature 23: note = K is covalently linked to the backbone
  modified_base 1..23: mod_base = OTHER; note = peptide nucleic acid
tccctccctccgttctttttccc

SEQ ID NO: 8 - DNA, 23 bp, Synthetic construct
  source 1..23: mol_type = other DNA; note = scramble gammaPNA
  misc_feature 1: note = TJJJJJJTJ-linker- is covalently linked to the backbone, wherein J is pseudoisocytosine and the linker is 11-Amino-3,6,9-Trioxaundecanoic Acid (DCHA), represented as -OOO-
  misc_feature 23: note = -K-linker-SEQ ID NO: 5 is covalently linked to the backbone, wherein the linker is 11-Amino-3,6,9-Trioxaundecanoic Acid (DCHA), represented as -OOO-
  modified_base 1..23: mod_base = OTHER; note = peptide nucleic acid
cctcccttcttccttctcccttg

SEQ ID NO: 9 - DNA, 23 bp, Synthetic construct
  source 1..23: mol_type = other DNA; note = gammaPNA
  misc_feature 1: note = JJJTJJJT-linker- is covalently linked to the backbone, wherein J is pseudoisocytosine and the linker is 11-Amino-3,6,9-Trioxaundecanoic Acid (DCHA), represented as -OOO-
  misc_feature 23: note = -K-linker-SEQ ID NO: 5 is covalently linked to the backbone, wherein the linker is 11-Amino-3,6,9-Trioxaundecanoic Acid (DCHA), represented as -OOO-
  modified_base 1..23: mod_base = OTHER; note = peptide nucleic acid
tccctcccctcccttctttttcc

SEQ ID NO: 10 - DNA, 24 bp, Synthetic construct
  source 1..24: mol_type = other DNA; note = gammaPNA
  misc_feature 1: note = JJJTJJJT-linker- is covalently linked to the backbone, wherein J is pseudoisocytosine and the linker is 11-Amino-3,6,9-Trioxaundecanoic Acid (DCHA), represented as -OOO-
  misc_feature 24: note = -K-linker-Label is covalently linked to the backbone, wherein the linker is 11-Amino-3,6,9-Trioxaundecanoic Acid (DCHA), represented as -OOO-, and the label is TAMARA
  modified_base 1..24: mod_base = OTHER; note = peptide nucleic acid
tccctccctccgttcttttttccc

SEQ ID NO: 11 - DNA, 24 bp, Synthetic construct
  source 1..24: mol_type = other DNA; note = gammaPNA to target
  misc_feature 1: note = JJJTJJJT-linker- is covalently linked to the backbone, wherein J is pseudoisocytosine and the linker is 11-Amino-3,6,9-Trioxaundecanoic Acid (DCHA), represented as -OOO-
  misc_feature 24: note = -K-linker-SEQ ID NO: 5-linker-label is covalently linked to the backbone, wherein the linker is 11-Amino-3,6,9-Trioxaundecanoic Acid (DCHA), represented as -OOO-, and the label is TAMARA
  modified_base 1..24: mod_base = OTHER; note = peptide nucleic acid
tccctccctccgttcttttttccc

SEQ ID NO: 12 - DNA, 64 bp, Synthetic construct
  source 1..64: mol_type = other DNA; note = primer
ctctgctttgggaacccgggaggggcgcttatggggagggtggggagggtggggaaggtgggga

SEQ ID NO: 13 - DNA, 64 bp, Synthetic construct
  source 1..64: mol_type = other DNA; note = primer
agagtgctcggctgcccggctgatgtctcttccccactccccaccttccccaccctccccaccc

SEQ ID NO: 14 - DNA, 62 bp, Synthetic construct
  source 1..62: mol_type = other DNA; note = primer
tcctgcctcgagaagggcagggcttctcagaggcttggcgggaaaaagaacggagggaggga

SEQ ID NO: 15 - DNA, 62 bp, Synthetic construct
  source 1..62: mol_type = other DNA; note = primer
agataaagccccgaaaaccggcttttatactcagcgcgatccctccctccgttctttttccc

SEQ ID NO: 16 - DNA, 23 bp, Homo sapiens
  source 1..23: mol_type = genomic DNA
tccctccctccgttctttttccc